(19) United States
(12) Patent Application Publication
(10) Pub. No.: US 2026/0345144 A1
(43) Pub. Date: Nov. 5, 2026

(54) IMPLANTABLE OCCLUDER DEVICES FOR TREATING ATRIAL SEPTAL DEFECTS

(71) Applicant: atHeart Medical AG, Baar (CH)

(72) Inventors: Stefano BUZZI, Birmensdorf (CH); Simone ESPOSITO, Winterthur (CH); Kuno LIMACHER, Steinhausen (CH); Fabien LOMBARDI, Lucerne (CH); Stefan MARTY, Steinhausen (CH); Andreas MELLMANN, Sigmaringen (DE); Claudio STEINER, Baar (CH)

(73) Assignee: atHeart Medical AG, Baar (CH)

(21) Appl. No.: 19/662,485

(22) Filed: Apr. 29, 2026

Related U.S. Application Data
(60) Provisional application No. 63/797,392, filed on Apr. 30, 2025.

(51) Int. Cl. A61B 17/00; A61B 90/00

(52) U.S. Cl. CPC A61B 17/0057; A61B 90/03; A61B 90/39; A61B 2017/00004; A61B 2017/00477; A61B 2017/00597; A61B 2017/00606; A61B 2017/00619; A61B 2017/00623; A61B 2090/034; A61B 2090/3966

(57) ABSTRACT

Implantable occluder devices, e.g., an atrial septal defect occluder having a bioresorbable frame, having reduced stiffness for implantation at the atrial septum of a patient are provided. The occluder may transition between a collapsed delivery state and an expanded deployed state where the proximal and distal portions of the occluder defined by a plurality of filaments extending between proximal and distal ends of the occluder expand radially outward to form disk-like structures having rounded outer edges. The proximal and distal ends of the occluder may be locked together to maintain the occluder in the expanded deployed state. Biocompatible fabric patches may be disposed over at least the proximal and distal portions, such that the patches fold over the rounded outer edges of the disk-like structures to thereby reduce trauma to the surrounding anatomy upon implantation.

### CROSS-REFERENCE TO RELATED APPLICATIONS

This application claims priority to U.S. Provisional Patent Application No. 63/797,392, filed Apr. 30, 2025, the entire contents of which are incorporated herein by reference.

### FIELD OF USE

This technology is directed to occluder devices having reduced stiffness for implantation at the atrial septum, particularly in patients with heart pathologies such as atrial septal defects (ASD).

### BACKGROUND

An atrial septal defect (ASD) is a congenital heart defect that can occur when the septum, the wall that separates the left and right atria in the heart, does not form or close properly. Commonly referred to as “hole in the heart,” an ASD causes oxygen-rich blood to mix in the oxygen-poor chamber, which would not otherwise occur. This increases blood flow to the lungs and over time, may lead to high blood pressure, arrhythmia, heart failure or an increased risk of stroke. In certain clinical applications, an ASD may be intentionally created to facilitate transseptal access to the left atrium for performing left-heart interventions, such as treating mitral valve defects. In these instances, the term “iatrogenic ASD” (iASD) is commonly employed to describe the created communication. For this disclosure, the terms “ASD” and “iASD” are used interchangeably and generally refer to any aperture or opening in the atrial septum, regardless of its etiology or origin.

ASDs vary in size. Some ASDs may close on their own early in life, while others require an intervention. Today, the vast majority are performed through minimally invasive catheter-based interventions, which consist of implanting an atrial septal occluder through a transcatheter procedure to close the defect. Current ASD occluder devices have dense metal frames that permanently clamp the septum. The long-term presence of metal in the heart may lead to potential complications and may limit future interventions that require crossing the interatrial septum.

U.S. Pat. No. 6,488,706 discloses a well-working implant for occluding a passage in a circulatory system. This implant has its particular application as a cardiological implant by means of which it is possible, for example, to close an aperture through the atrial septum or the ventricle septum of a heart. It is arranged to be introduced with a catheter and deployed or built up at a desired location in the body. This occluder comprises a plurality of thin wire-like elongate members which form a fixation structure and a single occluding body, namely a disk-like membrane, which is expanded by the fixation structure in the body passage. The fixation structure is locked in its expanded state by a locking unit. This locking unit comprises two locking elements which are brought together when the occluding body is expanded.

WO 2005/074813 discloses an occluder working with the same principle but comprising two occluding bodies which are expanded by the fixation structures, to thereby securely close both sides of the passage. WO 2012/156415 discloses a similar occluder that further comprises a jacket which envelops at least a part or a portion of the expanding unit so that no parts of the occluder can wander within the human or animal body. This jacket also enhances the growth of the body tissue over the occluder. WO 2010/151509, the entire contents of which are incorporated herein by reference, discloses an occluder for repair of cardiac and vascular defects or tissue openings that comprises an expandable frame consisting of elastic wires and a film encapsulating the expandable wire frame to enhance cellular growth. These above mentioned occluders form with their twisting elongate members and fixation structures, which permits the occluders to be opened and closed at a pace given by the user. Accordingly, the location of the occluder within the body passage can be changed until the fixation structure is locked in its expanded form by the locking unit.

An improved ASD occluder device is described in U.S. Pat. No. 10,820,907 to Mellmann et al., the entire contents of which are incorporated herein by reference. In view of the foregoing drawbacks of previously known systems and methods, there exists a need for further improved ASD occluder devices.

### SUMMARY

The present disclosure overcomes the drawbacks of previously-known systems and methods by providing an implantable occluder device for occluding a passage in a body, e.g., at an atrial septum of a patient. In accordance with some embodiments, an occluder device for occluding a passage in a body is provided. The occluder device may include a plurality of filaments coupled to and extending between proximal and distal ends of the occluder device to define a proximal portion, a distal portion, and a central portion disposed between the proximal and distal portions, the proximal and distal ends configured to move relative to each other to cause the plurality of filaments to transition between a collapsed delivery state and an expanded deployed state where the proximal and distal portions expand radially outward. In addition, the occluder device may include a biocompatible fabric patch configured to encapsulate at least the outer edges of the proximal and distal portions, the biocompatible fabric patch comprising a first inner membrane disposed between the proximal portion and the central portion, a second inner membrane disposed between the distal portion and the central portion, and an outer membrane disposed at the proximal and distal portions, each of the first and second inner membranes comprising a plurality of openings sized and shaped to receive the plurality of filaments therethrough. Moreover, the proximal and distal ends may be configured to lock together in a locked state to maintain the occluder device in the expanded deployed state.

The proximal portion may be configured to be disposed within a first atrium, the central portion may be configured to be disposed within an opening of an atrial septum, and the distal portion may be configured to be disposed within a second atrium such that, in the expanded deployed state, the proximal and distal portions sandwich the atrial septum. Moreover, the plurality of openings may be circumferentially disposed along each of the first and second inner membranes. The plurality of filaments may be arranged in a helical pattern about a longitudinal axis of the occluder device between the proximal and distal ends. Additionally, the plurality of filaments may be bioresorbable. In some embodiments, at least one of the outer membrane, the first inner membrane, or the second inner membrane may be formed of a bioresorbable material. Moreover, the occluder device further may include a plurality of radiopaque markers disposed adjacent to the plurality of openings. For example, the plurality of radiopaque markers may be coupled to at least one of the plurality of filaments, the first inner membrane, or the second inner membrane. In some embodiments, the plurality of radiopaque markers may be disposed on both sides of the plurality of openings of at least one of the first or second inner membranes in an alternating manner.

In addition, the proximal end may be configured to be removably coupled to an engagement portion at a distal region of an outer shaft of a delivery system, and the distal end may be configured to be removably coupled to an engagement portion at a distal region of an inner shaft slidably disposed within the outer shaft, such that relative movement between the distal regions of the inner and outer shafts causes the occluder device to transition between the collapsed delivery state and the expanded deployed state. Further, the proximal end and the distal end may be configured to be reattachably coupled to the distal regions of the outer shaft and the inner shaft, respectively, in situ. In some embodiments, a proximal region of the distal end may include an elongated portion comprising a protrusion, and a distal region of the proximal end may include an elongated portion comprising a counterbore extending circumferentially along an inner surface of a lumen of the proximal end, the counterbore configured to releasably receive the protrusion of the distal end in the locked state via a snap fit connection. For example, in the locked state, the proximal and distal ends may be separated by a predetermined distance configured to prevent the outer edges of the proximal and distal portions from flaring to thereby reduce tension in the occluder device in the expanded deployed state by reducing recoiled force generated by compression of the plurality of filaments in the expanded deployed state.

In some embodiments, a proximal region of the proximal end may include a stopper extending radially outwardly and circumferentially about the outer surface of the proximal end and a plurality of radially outwardly extending protrusions disposed circumferentially along the outer surface of the proximal end distal to the stopper, the plurality of radially outwardly extending protrusions separated by a plurality of openings extending between the outer surface of the proximal end and a lumen of the proximal end. The plurality of openings may be sized and shaped to releasably receive the engagement portion of the outer shaft when the engagement portion of the outer shaft is inserted through the lumen of the proximal end and aligned with the plurality of openings to thereby removably couple the proximal end to the outer shaft, such that the stopper prevents proximal movement of the outer shaft relative to the proximal end and the plurality of openings prevent distal movement of the outer shaft relative to the proximal end.

Accordingly, the engagement portion of the outer shaft may include a plurality of longitudinally extending arms configured to bend radially inward from a non-bended state for insertion through the lumen of the proximal end, each of the longitudinally extending arms comprising a distal protrusion configured to be releasably received through the plurality of openings of the proximal end in the non-bended state to thereby removably couple the proximal end to the outer shaft. Further, a lumen of the outer shaft may be sized and shaped to slidably receive a core therethrough, the core configured to prevent the plurality of longitudinally extending arms from bending radially inward from the non-bended state. For example, the core may be the inner shaft.

In some embodiments, a lumen of the distal end may include a counterbore extending circumferentially along an inner surface of the lumen, the counterbore sized and shaped to releasably receive the engagement portion of the inner shaft when the engagement portion of the inner shaft is inserted through the lumen of the distal end and aligned with the counterbore to thereby removably couple the distal end to the inner shaft. Additionally, a proximal side of the counterbore may have a tapered profile configured to facilitate decoupling of the engagement portion of the inner shaft from the distal end. Moreover, the engagement portion of the inner shaft may include a plurality of longitudinally extending arms configured to bend radially inward from a non-bended state for insertion through the lumen of the distal end, each of the longitudinally extending arms comprising a distal protrusion configured to be releasably received within the counterbore of the distal end in the non-bended state to thereby removably couple the distal end to the inner shaft. Further, a lumen of the inner shaft may be sized and shaped to slidably receive a core therethrough, the core configured to prevent the plurality of longitudinally extending arms from bending radially inward from the non-bended state. For example, the core may comprise a guidewire tube or a guidewire. Accordingly, when the core is disposed within the lumen of the inner shaft, a proximal side of the counterbore prevents proximal movement of the inner shaft relative to the distal end and a distal side of the counterbore prevents distal movement of the inner shaft relative to the distal end.

Alternatively, in some embodiments, the engagement portion of the inner shaft may include a plurality of flaps disposed circumferentially along an outer surface of the engagement portion proximal to the plurality of longitudinally extending arms, the plurality of flaps configured to transition between a collapsed position where the plurality of flaps extend parallel to a longitudinal axis of the inner shaft, and an expanded position where free distal ends of the plurality of flaps extend radially outward in the distal direction. The plurality of flaps may be biased towards the expanded position. Accordingly, when the core is disposed within the lumen of the inner shaft, a proximal side of the counterbore prevents proximal movement of the inner shaft relative to the distal end, and engagement between the plurality of flaps in the expanded position and a proximal side of the distal end prevents distal movement of the inner shaft relative to the distal end.

In some embodiments, the engagement portion of the inner shaft may include an outer tube comprising a plurality of longitudinally extending slits, and an inner tube slidably disposed within a lumen of the outer tube. For example, the inner tube may include a plurality of distal flaps disposed circumferentially along an outer surface of the inner tube, the plurality of distal flaps configured to transition between a collapsed position where the plurality of distal flaps extend parallel to a longitudinal axis of the inner shaft, and an expanded position where free proximal ends of the plurality of distal flaps extend radially outward through the plurality of slits in the proximal direction. The plurality of distal flaps may be biased towards the expanded position. Additionally, the inner tube further may include a plurality of proximal flaps disposed circumferentially along an outer surface of the inner tube proximal to the plurality of distal flaps, the plurality of proximal flaps configured to transition between a collapsed position where the plurality of proximal flaps extend parallel to a longitudinal axis of the inner shaft, and an expanded position where free distal ends of the plurality of proximal flaps extend radially outward through the plurality of slits in the distal direction. The plurality of proximal flaps may be biased towards the expanded position.

Accordingly, movement of the outer tube proximally relative to the inner tube causes the plurality of distal flaps to transition towards the collapsed position within the outer tube, and movement of the outer tube distally relative to the inner tube causes the plurality of proximal flaps to transition towards the collapsed position within the outer tube. Moreover, when the plurality of distal flaps are disposed within the counterbore of the distal end in the expanded position, a proximal side of the counterbore prevents proximal movement of the inner tube relative to the distal end, and engagement between the plurality of proximal flaps in the expanded position and a proximal side of the distal end prevents distal movement of the inner tube relative to the distal end. Further, when the core is disposed within the lumen of the inner shaft, a proximal side of the counterbore prevents proximal movement of the inner shaft relative to the distal end and a distal side of the counterbore prevents distal movement of the inner shaft relative to the distal end.

In some embodiments, a proximal region of the proximal end may include a proximal stopper extending radially outwardly and circumferentially about the outer surface of the proximal end, a distal stopper extending radially outwardly and circumferentially about the outer surface of the proximal end, and a gap disposed between the distal stopper and the proximal stopper. The gap may be configured to releasably receive the engagement portion of the outer shaft to thereby removably couple the proximal end to the outer shaft, such that, when the engagement portion of the outer shaft is received within the gap, the proximal stopper prevents proximal movement of the outer shaft relative to the proximal end and the distal stopper prevents distal movement of the outer shaft relative to the proximal end. Moreover, the proximal end further may include a plurality of planar surfaces disposed circumferentially about the outer surface of the proximal end within the gap, the plurality of planar surfaces configured to facilitate transmission of torque from the engagement portion of the outer shaft to the proximal end when engagement portion of the outer shaft is received within the gap.

Accordingly, the engagement portion of the outer shaft may include a plurality of longitudinally extending arms configured to bend radially outward from a non-bended state for advancement over the proximal stopper of the proximal end, each of the longitudinally extending arms comprising a distal hook portion comprising a tooth configured to be received within the gap and a receptacle configured to receive at least a portion of the proximal stopper therein to thereby removably couple the proximal end to the outer shaft. The plurality of longitudinally extending arms may be configured to be constrained in the non-bended state via an external sleeve slidably disposed over the engagement portion of the outer shaft.

In some embodiments, the outer membrane may comprise a continuous outer cover configured to be disposed over the proximal portion, the central portion, and the distal portion. The continuous outer cover may only be fixed to the occluder device via the proximal and distal ends of the occluder device. For example, the proximal and distal ends of the outer cover may be configured to be coupled to the occluder device via at least one of mechanical crimping, chemical bonding, thermal bonding, or suturing. Moreover, the outer cover may be formed by a thin weaved tubing, a knitted fabric, or an electro-spun polymer. For example, the electro-spun polymer may include polytetrafluoroethylene (PTFE), polyethylene terephthalate (PET), or polyurethane (PU). In some embodiments, the outer cover may be configured to promote cell growth.

Additionally, at least one of the first or second inner membranes may include a central aperture and a plurality of hollowed openings disposed circumferentially about the central aperture, the plurality of hollowed openings separated by a plurality of radially extending spokes and configured to improve membrane crossability. Moreover, the occluder device may include a plurality of radiopaque markers disposed adjacent to the plurality of openings, the plurality of radiopaque markers arranged to facilitate visualization of at least one recross zone sized and shaped to improve membrane crossability. For example, the at least one recross zone may be aligned with at least one hollowed opening of the plurality of hollowed openings. Additionally or, alternatively, at least one of the first or second inner membranes comprise a full disk shape having a central aperture.

Alternatively, in some embodiments, the outer membrane may include a first outer membrane disposed on the proximal facing side of the proximal portion, an outer edge portion of the first outer membrane configured to be coupled to the outer perimeter of the first inner membrane, and a second outer membrane disposed on the distal facing side of the distal portion, an outer edge portion of the second outer membrane configured to be coupled to the outer perimeter of the second inner membrane. The first and second outer membranes may be coupled to the first and second inner membranes at a location radially inward from the outer edges of the proximal and distal portions, respectively, to reduce trauma to surrounding anatomy. In some embodiments, only a first subset of the plurality of filaments may be coupled to the first outer membrane at the proximal portion, and only a second subset of the plurality of filaments may be coupled to the second outer membrane at the distal portion to thereby reduce global stiffness of the occluder device.

Moreover, the first and second inner membranes may be sized and shaped to fold over the outer edges of the proximal and distal portions, respectively, such that an outer perimeter of the first inner membrane is disposed on a proximal facing side of the proximal portion and an outer perimeter of the second inner membrane is disposed on a distal facing side of the distal portion. Additionally, each of the first and second inner membranes may include a plurality of tabs disposed circumferentially along its outer perimeter, the plurality of tabs configured to extend radially inward. Further, each of the plurality of tabs may include a plurality of guiding holes sized and shaped to facilitate suturing of the first and second inner membranes to the first and second outer membranes, respectively. Alternatively, in some embodiments, the first and second outer membranes may be sized and shaped to fold over the outer edges of the proximal and distal portions, respectively, such that an outer perimeter of the first outer membrane is disposed on a distal facing side of the proximal portion and an outer perimeter of the second outer membrane is disposed on a proximal facing side of the distal portion.

### BRIEF DESCRIPTION OF THE DRAWINGS

FIGS. 1A to 1C illustrate an implantable occluder device.

FIGS. 2A to 2C illustrate an exemplary implantable occluder device having reduced stiffness constructed in accordance with some embodiments.

FIGS. 3A to 3E illustrate exemplary outer and inner membranes of the biocompatible fabric patches of the implantable occluder device of FIGS. 2A to 2C, and assembly thereof.

FIG. 3F illustrates alternative exemplary outer and inner membranes of a biocompatible fabric patch of the implantable occluder device of FIGS. 2A to 2C.

FIG. 4 illustrates an alternative exemplary implantable occluder device having biocompatible fabric patches formed of outer and inner membranes in accordance with some embodiments.

FIGS. 5A and 5B illustrate the snap fit connection between the proximal and distal ends of the implantable occluder device of FIGS. 1A to 1C.

FIGS. 6A and 6B illustrate the snap fit connection between the exemplary proximal and distal ends of the implantable occluder device of FIGS. 2A to 2C in accordance with some embodiments.

FIGS. 7A to 7C illustrate an alternative exemplary implantable occluder device having a biocompatible fabric patch including exemplary wagon wheel shaped inner membranes and a continuous outer covering constructed in accordance with some embodiments.

FIGS. 7D and 7E illustrate the implantable occluder device of FIGS. 7A to 7C in a collapsed state.

FIG. 7F illustrates transitioning of the implantable occluder device of FIGS. 7A to 7C from an expanded state to a collapsed state.

FIG. 7G illustrates an exemplary recross zone of the implantable occluder device of FIGS. 7D and 7E in the collapsed state.

FIG. 7H shows photographs of the implantable occluder device of FIG. 7A including an outer cover formed of a knitted fabric in accordance with some embodiments.

FIGS. 8A to 8E illustrate exemplary method steps for implanting the implantable occluder device of FIGS. 7A to 7G in accordance with some embodiments.

FIGS. 9A to 9C illustrate the implantable occluder device of FIGS. 7A to 7G implanted an at atrial septal defect after release from the delivery system, during interim degradation of the filaments of the occluder device, and after degradation of the filaments, respectively.

FIG. 9D is a side view of the implantable occluder device of FIGS. 7A to 7G implanted an at atrial septal defect after degradation of the filaments.

FIG. 10A illustrates another alternative exemplary implantable occluder device having a biocompatible fabric patch including exemplary full disk shaped inner membranes and an outer covering constructed in accordance with some embodiments.

FIG. 10B illustrates the full disk shaped inner membranes of the biocompatible fabric patch of the implantable occluder device of FIG. 10A.

FIG. 11 illustrates alternative exemplary inner membranes of the biocompatible fabric patch comprising a wagon wheel shaped inner membrane and a full disk shaped inner membrane in accordance with some embodiments.

FIG. 12 illustrates additional alternative exemplary inner membranes of the biocompatible fabric patch comprising ring shaped inner membranes in accordance with some embodiments.

FIGS. 13A to 13E illustrate an alternative exemplary coupling mechanism for removably coupling the outer and inner shafts of a delivery system to the proximal and distal ends of an implantable occluder device in accordance with some embodiments.

FIGS. 14A to 14C illustrate an alternative exemplary coupling mechanism for removably coupling the inner shaft of a delivery system to the distal end of an implantable occluder device in accordance with some embodiments.

FIGS. 15A to 15C illustrate another alternative exemplary coupling mechanism for removably coupling the outer shaft of a delivery system to the proximal end of an implantable occluder device in accordance with some embodiments.

FIGS. 16A and 16B illustrate another alternative exemplary coupling mechanism for removably coupling the inner shaft of a delivery system to the distal end of an implantable occluder device in accordance with some embodiments.

### DETAILED DESCRIPTION

The present disclosure is directed to improved occluder devices having reduced stiffness and/or smoother, less abrasive outer edges for implantation at the atrial septum of a patient's heart for treating patients suffering from an atrial septal defect (ASD). The low-profile, implantable ASD occluder devices described herein have a metal-free, bioresorbable frame designed for the closure of native or iatrogenic atrial septal defects. For example, the low-profile occluder devices may include bioresorbable filaments connecting one or more biocompatible fabric (e.g., woven or non-woven) patches, which may contain radiopaque markers. For example, the fabric patches may be formed of polyester (e.g., polyethylene terephthalate (PET)), an electro-spun (e-spun) polymer membrane (e.g., polytetrafluoroethylene (PTFE) or polyurethane (PU)), or a combination of both.

The ASD occluder devices may be advanced in a collapsed delivery state over a guidewire to the atrial septum via a delivery system such as those described in WO 2025/083644 A1, the entire contents of which are incorporated herein by reference, and may be fully deployed at the atrial septum with the guidewire in place, providing the opportunity to reattach and reposition the occluder device if necessary. After endothelialization, the bioresorbable filaments slowly resorb, with complete resorption within a predetermined timeframe, e.g., within 24 months, such that the biocompatible fabric and the radiopaque markers remain at the atrial septum, which may be useful for future transseptal procedure planning.

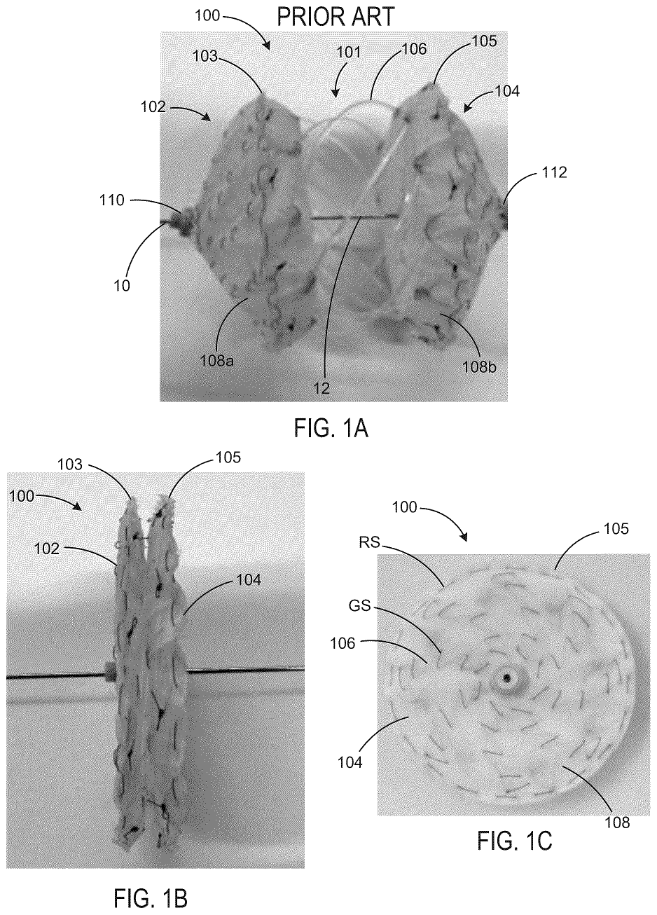

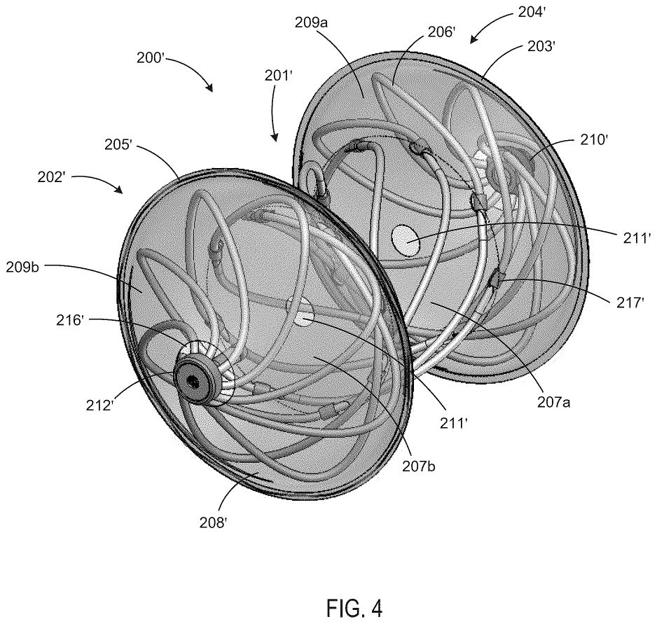

As shown in FIGS. 1A to 1C, an implantable occluder device, e.g., implantable occluder device 100, may include a plurality of wire-like filaments 106 extending between proximal end 110 and distal end 112 of occluder 100. Proximal end 110 may be configured to removably engage with an engagement portion at the distal region of outer shaft 10 of a delivery system, as described in WO 2025/083644 A1, and distal end 112 may be configured to removably engage with an engagement portion at the distal region of inner shaft 12 slidably disposed within outer shaft 10, such that relative motion between outer shaft 10 and inner shaft 12 and, accordingly, proximal end 110 and distal end 112 of occluder 100 transitions occluder device 100 between a collapsed delivery state and an expanded deployed state, as shown in FIG. 1B. FIG. 1A illustrates occluder 100 in a partially expanded state. Proximal end 110 and distal end 112 of occluder 100 may be locked together when occluder 100 is in its expanded deployed state, e.g., when proximal end 110 is moved towards and engages with distal end 112 via distal movement of outer shaft 10 relative to inner shaft 12, to thereby lock occluder 100 in its full expanded deployed state, as described in further detail below, with regard to FIGS. 4A and 4B.

Filaments 106 may be fixedly coupled to proximal end 110 at its proximal end and fixedly coupled to distal end 112 at its distal end, and may define proximal portion 102, central portion 101, and distal portion 104 of occluder 100. Filaments 106 may be arranged in a helical pattern about the longitudinal axis of occluder 100 between proximal end 110 and distal end 112, such that, when occluder 100 is in its collapsed delivery state where proximal end 110 and distal end 112 are spaced apart, e.g., by a predetermined distance, proximal portion 102, central portion 101, and distal portion 104 of occluder 100 all have an elongated configuration and are radially contracted towards the longitudinal axis of occluder 100, and when occluder 100 is in its expanded deployed state where proximal end 110 and distal end 112 are moved adjacent to one another, proximal portion 102 and distal portion 104 expand radially outward, as shown in FIG. 1B.

In addition, occluder 100 may include biocompatible fabric patches, e.g., proximal patch 108a and distal patch 108b (collectively referred to herein as patches 108), disposed on at least proximal portion 102 and distal portion 104, and connected via filaments 106. For example, as shown in FIG. 1A, proximal patch 108a may be sutured to filaments 106 at proximal portion 102, e.g., via guiding sutures GS along the proximal facing side of proximal patch 108a, and distal patch 108b may be sutured to filaments 106 at distal portion 104, e.g., via guiding sutures GS along the distal facing side of distal patch 108b. Generally, each individual filament of filaments 106 may be sutured to the respective proximal and distal patches, as shown in FIG. 1C, which illustrates eight individual filaments. Moreover, guiding sutures GS may suture filaments 106 to patches 108 in a manner that permits filaments 106 to slide relative to patches 108 as occluder 100 transitions between the collapsed delivery state and the expanded deployed state. Additionally, each patch 108 is generally formed of an inner membrane and an outer membrane that are sutured together along their respective outer edges via running suture RS.

As shown in FIG. 1B, as occluder 100 transitions from the collapsed delivery state to the expanded deployed and locked state, filaments 106 cause patches 108 to form disk-like structures at proximal portion 102 and distal portion 104, defining outer edge 103 at proximal portion 102 and outer edge 105 distal portion 104, such that proximal portion 102 and distal portion 104 may sandwich the atrial septum in the expanded deployed and locked state. Accordingly, in the expanded deployed state, running sutures RS that couple the inner and outer membranes of each patch 108 run along the respective outer edges, e.g., outer edge 103 of proximal portion 102 and outer edge 105 of distal portion 105, as shown in FIG. 1C.

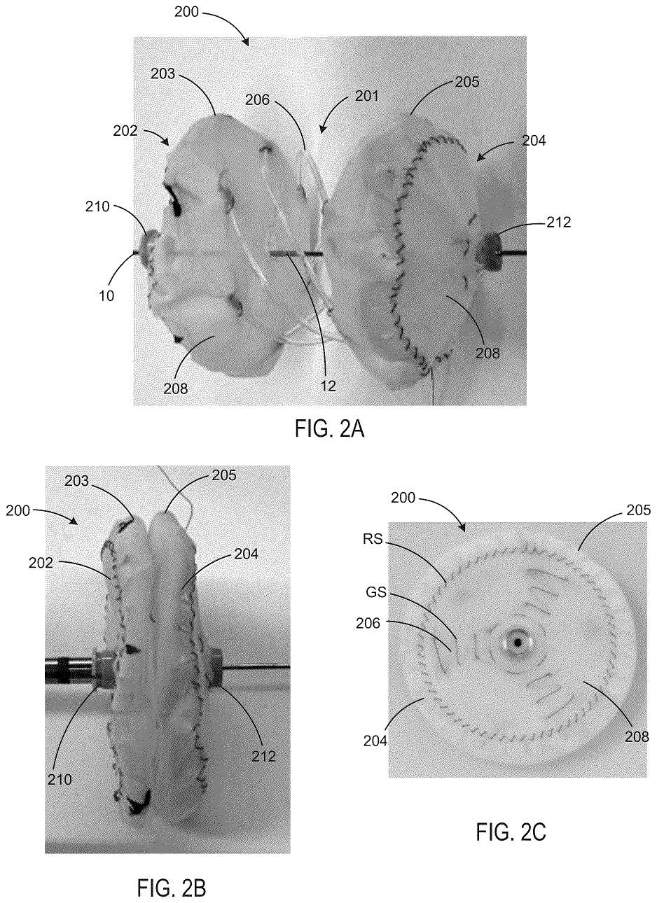

Referring now to FIGS. 2A to 2C, an exemplary implantable occluder device having reduced stiffness is provided. As shown in FIG. 2A, occluder 200 may include a plurality of bioresorbable wire-like filaments, e.g., filaments 206, extending between proximal end 210 and distal end 212 of occluder 200. Like proximal end 110 of occluder 100, proximal end 210 may be configured to removably and reattachably engage with an engagement portion at the distal region of outer shaft 10. For example, the distal region of outer shaft 10 may include a threaded surface, e.g., disposed on an inner surface of the lumen of outer shaft 10, and the outer surface of proximal end 210 may have a threaded surface configured to removably and reattachably engage with the threaded surface of outer shaft 10, e.g., via relative rotation between outer shaft 10 and proximal end 210, or vice versa (e.g., the threaded surface of proximal end 210 may be disposed on an inner surface of proximal end 210 for removable engagement with a threaded outer surface at the distal region of the outer shaft). Moreover, the distal region of inner shaft 12 may include a threaded surface, e.g., disposed on an outer surface at the distal region of inner shaft 12, and at least a portion of the inner surface of the lumen of distal end 212 may have a threaded surface configured to removably and reattachably engage with the threaded surface of inner shaft 12, e.g., via relative rotation between inner shaft 12 and distal end 212. Accordingly, the lumen of distal end 212 may be sized and shaped to receive inner shaft 12 therethrough.

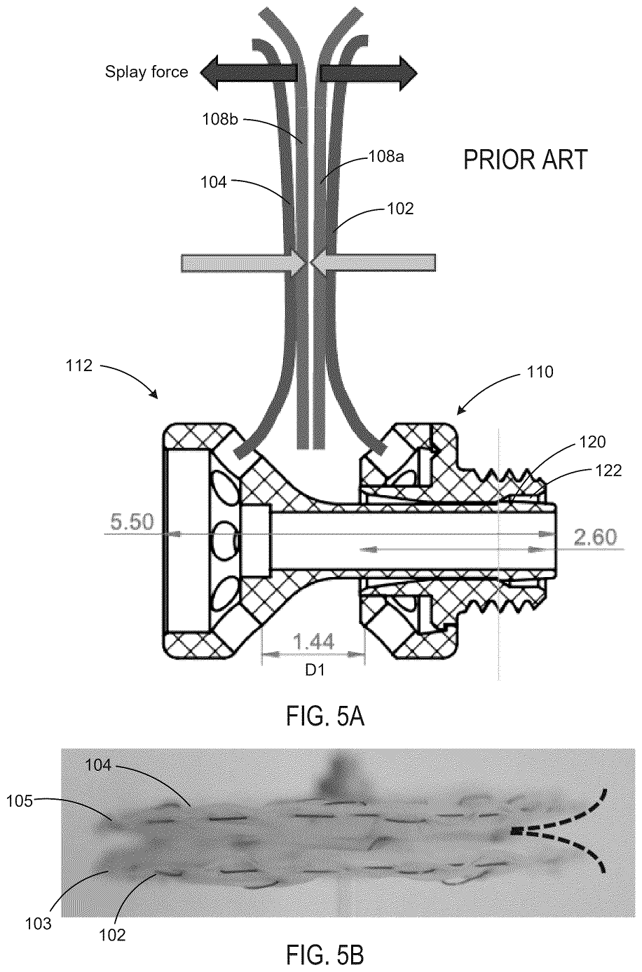

When outer shaft 10 is coupled to proximal end 210 of occluder 200 and inner shaft 12 is coupled to distal end 212 of occluder 200, relative movement between outer shaft 10 and inner shaft 12 will cause occluder 200 to transition between its collapsed delivery state and its expanded deployed state. For example, the distal end of outer shaft 10 may be spaced apart from the distal end of inner shaft 12 by a distance such that occluder 200 contracts radially inward and has an elongated configuration in the collapsed delivery state. In the collapsed delivery state, occluder 200 may have an outer diameter sized to fit within the lumen of a delivery sheath, as described in WO 2025/083644 A1. Moreover, the distal end of outer shaft 10 may be positioned adjacent to the distal end of inner shaft 12 such that proximal portion 202 and distal portion 204 of occluder 200 expand radially outward in the expanded deployed state. In addition, proximal end 210 and distal end 212 of occluder 200 may be locked together when occluder 200 is in its expanded deployed state, e.g., when proximal end 210 is moved towards and engages with distal end 212 via distal movement of outer shaft 10 relative to inner shaft 12, to thereby lock occluder 200 in its full expanded deployed state, as described in further detail below, with regard to FIGS. 5A and 5B.

Referring again to FIG. 2A, filaments 206 may be fixedly coupled to proximal end 210 at its proximal end and fixedly coupled to distal end 210 at its distal end, and may define proximal portion 202, central portion 201, and distal portion 204 of occluder 200. Filaments 206 may be arranged in a manner such that, when occluder 200 is in its collapsed delivery state where proximal end 210 and distal end 212 are spaced apart, e.g., by a predetermined distance, proximal portion 202, central portion 201, and distal portion 204 of occluder 200 all have an elongated configuration and are radially contracted towards the longitudinal axis of occluder 200. Moreover, when occluder 200 is in its expanded deployed state where proximal end 210 and distal end 212 are moved adjacent to one another, proximal portion 202 and distal portion 204 expand radially outward, as shown in FIG. 2B. For example, filaments 206 may be arranged in a helical pattern about the longitudinal axis of occluder 200 between proximal end 210 and distal end 212. Thus, when implanted at the atrial septum, central portion 201 may extend across the atrial septal defect, such that distal portion 204 is deployed within a first atrium, e.g., the left atrium, and proximal portion 202 is deployed within a second atrium, e.g., the right atrium.

In addition, occluder 200 may include biocompatible fabric (e.g., woven or non-woven) patches, e.g., proximal patch 208a and distal patch 208b (collectively referred to herein as patches 208), disposed on at least proximal portion 202 and distal portion 204, and connected via filaments 206. For example, proximal patch 208a may be sutured to one or more filaments 206 at proximal portion 202, e.g., via guiding sutures GS along the proximal facing side of proximal patch 208a, and distal patch 208b may be sutured to one or more filaments 206 at distal portion 204, e.g., via guiding sutures GS along the distal facing side of distal patch 208b, in a manner that permits filaments 206 to slide relative to patches 208 as occluder 200 transitions between the collapsed delivery state and the expanded deployed state. In addition, filaments 206 may include one or more radiopaque markers disposed thereon, such that occluder 200 may be visualized under fluoroscopy. Unlike patches 108 of occluder 100, not every individual filament of filaments 206 may be sutured to the respective proximal and distal patches. For example, as shown in FIG. 2C, which illustrates six individual filaments, only three individual filaments, e.g., every other filament, may be sutured to the respective patch 108. Accordingly, occluder 200 requires fewer guiding sutures GS to suture patches 208 to filaments 206, thereby permitting more degrees of freedom between the components of occluder 200 and reducing the overall global stiffness of occluder 200. As will be understood by a person having ordinary skill in the art, fewer or more than half of the total number of individual filaments may be sutured to the respective patch, while still reducing overall stiffness of the occluder.

Patches 208 may be made from a biocompatible material such as polyester (e.g., polyethylene terephthalate (PET)), an electrospun (e-spun) membrane (e.g., polyurethane (PU)), or a combination of both. In some embodiments, patches 208 may include one or more radiopaque markers disposed thereon, such that occluder 200 may be visualized under fluoroscopy. As shown in FIG. 2B, as occluder 200 transitions from the collapsed delivery state to the expanded deployed and locked state, filaments 206 cause patches 208 to form disk-like structures at proximal portion 202 and distal portion 204, defining outer edge 203 at proximal portion 202 and outer edge 205 distal portion 204, such that proximal portion 202 and distal portion 204 may sandwich the atrial septum in the expanded deployed and locked state. Like patches 108 of occluder 100, each patch 208 may be formed of first and second membranes, e.g., an inner membrane and an outer membrane, that are sutured together via running suture RS. Preferably, running suture RS may be a single continuous suture, and does not engage any of the filaments.

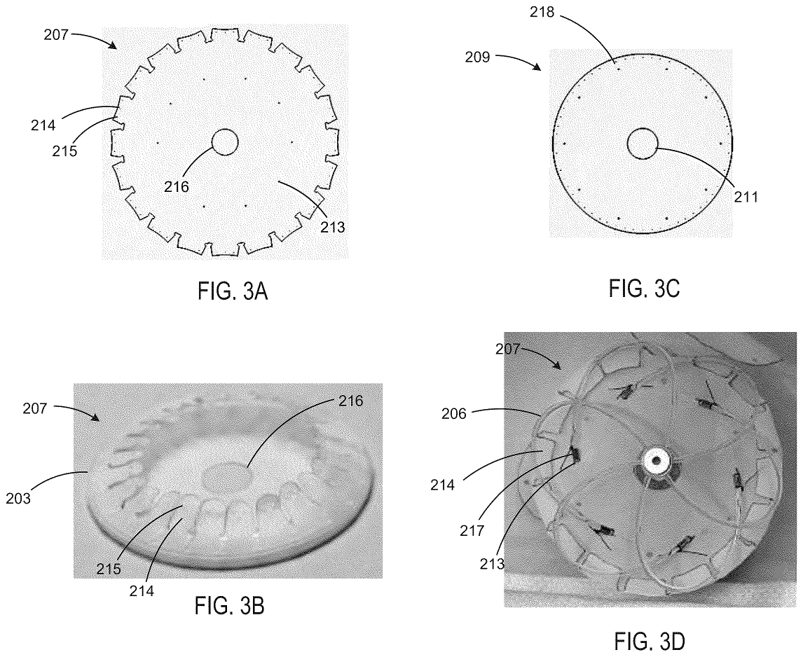

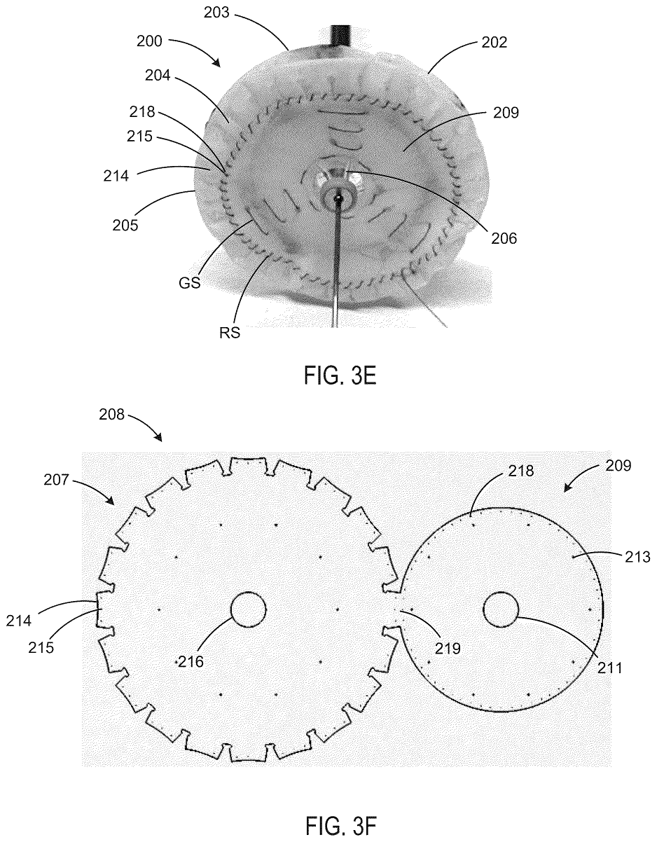

However, unlike patches 108, a first membrane, e.g., the inner membrane, of each patch 208 may be enlarged to fold over the respective outer edge, e.g., outer edge 203 of proximal portion 202 and outer edge 205 of distal portion 204, and sutured to the respective second membrane, e.g., the outer membrane, along the outer surface of occluder 200, e.g., the proximal facing surface of proximal portion 202 and the distal facing surface of distal portion 204, as described in further detail below with regard to FIGS. 3A to 3F. Accordingly, by folding the inner membranes over the outer edges of respective proximal portion 202 and distal portion 204, in the expanded deployed state, filaments 206 are encapsulated along the outer perimeters of proximal portion 202 and distal portion 204, such that outer edges 203, 205 are less traumatic/abrasive on the surrounding anatomy when occluder 200 is implanted. Once implanted at the atrial septum, tissue ingrowth on occluder 200, i.e., endothelialization, further secures occluder 200 at the atrial septum and occludes the atrial septal defect. Moreover, filaments 206 may resorb over time, with complete resorption within, e.g., 24 months, such that patches 208 at proximal portion 202 and distal portion 204 and, accordingly, the radiopaque markers remain implanted at the atrial septum. Occluder 200 may be sized to support closure of defects ranging from, e.g., 8-28 mm.

Referring now to FIGS. 3A to 3F, exemplary components of patches 208, e.g., the inner and outer membranes, and assembly thereof are provided. As described above, each patch 208 may be formed of a first membrane (e.g., inner membrane 207), e.g., the membrane axially closer to central portion 201, and a second membrane (e.g., outer membrane 209), e.g., the membrane axially closer to proximal end 210 or distal end 212. Inner membranes 207 may be formed of a weaved fabric material and may be shape set via various techniques known in the art. For example, inner membranes 207 may be laser cut to define a plurality of tabs 214 extending along the outer perimeter of inner membranes 207, and to further define aperture 216 sized and shaped to permit at least inner catheter 12 to pass therethrough, as shown in FIG. 3A. Moreover, each tab 214 may include a plurality of guiding holes 215 sized and shaped to guide running suture RS during suturing of inner membrane 207 to outer membrane 209.

Inner membranes 207 further may include a plurality of guiding holes 213 disposed circumferentially around aperture 216, each guiding hole 213 sized and shaped to guide a filament 206 therethrough such proximal portion 202 of filaments 206 is disposed within proximal patch 208a and distal portion 204 of filaments 206 is disposed within distal patch 208b. Inner membranes 207 may be heat seat, e.g., via one or more heat set forming tools, to form the shape illustrated in FIG. 3B, such that the outer edge portion of inner membranes 207 is folded over to define the outer edge of the patch, e.g., outer edge 203 of proximal portion 202, and the plurality of tabs 214 face radially inward towards aperture 216. The cut pattern of inner membranes 207 may be optimized such that overlap between adjacent tabs 214 is minimized.

Outer membranes 209 also may be formed of a weaved fabric material and laser cut to define aperture 211 sized and shaped to permit at least inner shaft 12 and the ends of filaments 206 to pass therethrough, as shown in FIG. 3C. Additionally, the outer edge portion of outer membranes 209 may include guiding holes 218 sized and shaped to guide running suture RS during suturing of outer membrane 209 to inner membrane 207. Accordingly, guiding holes 218 may be aligned with guiding holes 215 when inner membrane 207 comprises the shape illustrated in FIG. 3B.

As shown in FIG. 3D, each of filaments 206 may be passed through a corresponding guiding hole 213 of inner membrane 207, such that the proximal and distal portions of filaments 206 having the maximum outer diameter in the expanded deployed state may be positioned between the inner and outer membranes of the respective patch when the inner and outer membranes are coupled together. Accordingly, the radially outermost edge of filaments 206 may further define the outer edges of occluder 200, e.g., outer edge 203 at proximal portion 202 and outer edge 205 at distal portion 204, in the expanded deployed state. As shown in FIG. 3D, a plurality of radiopaque markers 217 may be disposed, e.g., sewn, on inner membrane 207, e.g., at guiding holes 213, and/or on filaments 206, e.g., adjacent to guiding holes 213.

As shown in FIG. 3E, plurality of tabs 214 of inner membranes 207 may be folded over the radially outermost edge of filaments 206 at each of proximal portion 202 and distal portion 204 to define outer edge 203 and outer edge 205, respectively, and sutured to the outer edge portion of outer membranes 209, e.g., via running suture RS along guiding holes 213 and guiding holes 215, such that running suture RS runs along the outer surface of the respective patch, radially inward from the outer edge of the respective patch. Moreover, in some embodiments, as described above, only a subset of filaments 206 may be sutured to outer membrane 209, e.g., via guiding sutures GS, such that the filaments that are not sutured to outer membrane 209 are free to move relative to outer membrane 209 within the respective patch, thereby reducing the global stiffness of occluder 200.

FIG. 3F illustrates an alternative embodiment where the inner and outer membranes (e.g., inner membranes 207 and outer membranes 209) may be integrally formed as a single piece, and subsequently sutured together, as described above. For example, as shown in FIG. 3F, inner membrane 207 may be connected to outer membrane 209 at connection 219, such that outer membrane 209 may be folded over connection 219 to align aperture 211 with aperture 216, and guiding holes 213 with guiding holes 215, e.g., when plurality of tabs 214 are folded radially inward, as described above.

Referring now to FIG. 4, an alternative exemplary implantable occluder device is provided. Implantable occluder device 200′ may be constructed similar to implantable occluder device 200, with similar components having like-prime reference numerals. For example, the framework of occluder 200′ includes proximal end 210′, distal end 212′, and filaments 206′ extending therebetween, which correspond with proximal end 210, distal end 212, and filaments 206, and occluder 200′ further includes biocompatible fabric patches 208′, each formed by an inner membrane coupled to an outer membrane, which correspond with biocompatible fabric patches 208. Patches 208′ differs from patches 208 in that proximal and distal inner membranes 207a, 207b may be structured similar to outer membranes 209 and proximal and distal outer membranes 209a, 209b may be structured similar to inner membranes 207. For example, the plurality of tabs (not shown) at the outer edge portion of proximal and distal outer membranes 209a, 209b may be folded inward toward central portion 201′ over the radially outermost edges of filaments 206′ to define rounded outer edge 203′ at proximal portion 202′ and rounded outer edge 205′ at distal portion 204′, and sutured to the outer edge portion of proximal and distal inner membranes 207a, 207b, respectively, thereby similarly reducing global stiffness of occluder 200′.

Referring now to FIGS. 5A and 5B, locking of proximal end 110 and distal end 112 of occluder 100 in the expanded deployed state is provided. As shown in FIG. 5A, proximal end 110 may have a lumen extending therethrough sized and shaped to receive at least an elongated portion of distal end 112, and the lumen may comprise counterbore 122 extending circumferentially along the inner surface of a proximal portion of the lumen. Counterbore 122 may be sized and shaped to securely receive protrusion 120 of distal end 112, e.g., via a snap fit connection. Accordingly, counterbore 122 may have a geometry that corresponds with the geometry of protrusion 120, such that when protrusion 120 is disposed within counterbore 122, proximal end 110 is securely coupled to distal end 112 in a locked state, as shown in FIG. 5A, and occluder 100 is in its fully expanded deployed and locked state, as shown in FIG. 5B.

As shown in FIG. 5A, a distal portion of the lumen of proximal end 110 may be tapered to facilitate insertion of protrusion 120 therethrough, and protrusion 120 may have an outer diameter that is slightly larger than the portion of the lumen of proximal end 110 distal to counterbore 122, such that when protrusion 120 is disposed within counterbore 122, the inner wall of the lumen distal to counterbore 122 prevents distal movement of distal end 112 relative to proximal end 110. In the locked state, proximal end 110 is separated from distal end 112 by a predetermined distance D1, thereby minimizing the distance between proximal portion 102 and distal portion 104 and, accordingly, proximal patch 108a and distal patch 108b when occluder 200 is in the expanded deployed state. The compression of occluder 100 in the locked state causes the unconstrained outer edges of the filaments at proximal portion 102 and distal portion 104 to flare at outer edge 103 and outer edge 105, respectively, due to the maximal recoiled force generated by the compressed filaments, thereby resulting in a large splay force, as shown in FIG. 5A.

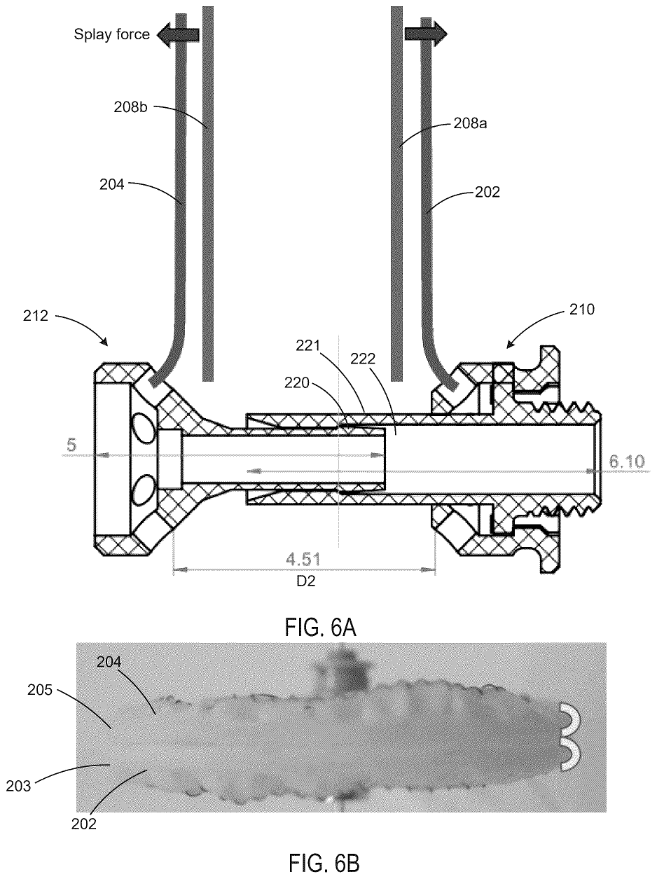

Referring now to FIGS. 6A and 6B, locking of proximal end 210 and distal end 212 of occluder 200 in the expanded deployed state is provided. Proximal end 210 and distal end 212 may be constructed similar to proximal end 110 and distal 112 of occluder 100. For example, as shown in FIG. 6A, proximal end 210 may have a lumen extending therethrough sized and shaped to receive at least an elongated portion of distal end 212, and the lumen may comprise counterbore 222 extending circumferentially along the inner surface of a proximal portion of the lumen. Counterbore 222 may be sized and shaped to securely receive protrusion 220 of distal end 212, e.g., via a snap fit connection. Accordingly, counterbore 222 may have a geometry that corresponds with the geometry of protrusion 220, such that when protrusion 220 is disposed within counterbore 222, proximal end 210 is securely coupled to distal end 212 in a locked state, as shown in FIG. 6A, and occluder 200 is in its fully expanded deployed and locked state, as shown in FIG. 6B. Moreover, as shown in FIG. 6A, a distal portion of the lumen of proximal end 210 may be tapered to facilitate insertion of protrusion 220 therethrough, and protrusion 220 may have an outer diameter that is slightly larger than the portion of the lumen of proximal end 210 distal to counterbore 222, such that when protrusion 220 is disposed within counterbore 222, the inner wall of the lumen distal to counterbore 222 prevents distal movement of distal end 212 relative to proximal end 210.

Unlike proximal end 110, proximal end 210 may include elongated portion 221 extending distally therefrom, e.g., toward distal end 212, such that counterbore 222 may be at least partially disposed within elongated portion 221. Accordingly, as shown in FIG. 6A, in the locked state, proximal end 210 is separated from distal end 212 by a predetermined distance D2, wherein D2 is larger than D1 of FIG. 5A, such that the compression of occluder 200 in the locked state does not cause the unconstrained outer edges of the filaments at proximal portion 202 and distal portion 204 to flare at outer edge 203 and outer edge 205, respectively, thereby reducing the tension in the framework of occluder 200 in the expanded deployed state, e.g., by reducing the recoiled force generated by the compressed filaments, resulting in a smaller splay force, as shown in FIG. 6A. For example, predetermined distance D2 may be selected such that the splay force generated on proximal portion 202 and distal portion 204 of occluder 200 may be reduced compared with occluder 100. Moreover, the splay force generated on proximal portion 202 and distal portion 204 of occluder 200 further may be reduced by suturing only a subset of filaments 206 to outer membranes 209, as described above, compared with suturing all of the individual filaments 106 to the outer membrane 109.

As shown in FIG. 6B, outer edge 203 of proximal portion 202 and outer edge 205 of distal portion 204 have a more rounded profile in the expanded deployed state compared to outer edge 103 of proximal portion 102 and outer edge 105 of distal portion 104, such that rounded outer edges 203, 205 are less traumatic/abrasive on the surrounding anatomy, e.g., the atrial septal tissue. In some embodiments, elongated portion 221 of proximal end 210 may define a plurality of arms joined together at the proximal end of elongated portion 221 and extending longitudinally towards the distal end of elongated portion 221, wherein each adjacent arm is separated by a longitudinally extending slit and comprises a grabber portion (e.g., a hook shaped portion) for engaging with protrusion 220. For example, the plurality of arms may include 2-4 arms separated via 2-4 slits. Accordingly, the plurality of arms defining elongated portion 221 may reduce plastic deformation and force during engagement with distal end 212, and further may reduce sensitivity on tight manufacturing tolerances. Alternatively, rather than elongated portion 221 being defined by a plurality of arms, at least the proximal portion of protrusion 220 of distal end 212 may similarly be defined by a plurality of arms. Moreover, while defining elongated portion 221 or protrusion 220 via a plurality of arms reduces force required to engage proximal end 210 with distal end 212, such a design further allows the force required to disengage proximal end 210 from distal end 212 to remain relatively high, e.g., by providing a steep interface angle (e.g., nearly 90°) between the distal side of protrusion 220 and the proximal side of the grabber portion of the plurality of arms of proximal end 210 (or the distal side of counterbore 222 when protrusion 220 is defined by a plurality of arms), as a safety feature. On the other hand, a flat interface angle (e.g., 30°) may reduce the unlocking force to allow for soft unlocking. Moreover, a flat interface angle (e.g., 15°) between the proximal side of protrusion 220 and the distal side of the grabber portion of the plurality of arms of proximal end 210 (or the distal side of elongated portion 221 when protrusion 220 is defined by a plurality of arms) may reduce the engagement force, while a steep interface angle (e.g., nearly 90°) may increase the engagement force.

Occluder 200 may be delivered to and deployed at the target anatomical site, e.g., an atrial septal defect, via a delivery system such as those described in WO 2025/083644 A1. For example, a guidewire initially may be advanced through the patient's body to the atrial septum, e.g., through the atrial septal defect. A delivery sheath having occluder 200 disposed therein in its collapsed delivery state may be advanced over the guidewire (e.g., via a guidewire lumen of inner shaft 12) to the patient's atrial septum, such that central portion 201 of occluder 200 is aligned with the atrial septum, distal portion 204 of occluder 200 is disposed within a first atrium, e.g., the left atrium, and proximal portion 202 of occluder 200 is disposed within a second atrium, e.g., the right atrium. The delivery sheath may then be retracted proximally relative to outer shaft 10, inner shaft 12, and occluder 200, e.g., via actuation at a handle operatively coupled to the delivery sheath, outer shaft 10, and inner shaft 12, to thereby expose distal portion 204 within the left atrium and proximal portion 202 within the right atrium. Alternatively, the delivery sheath may be separate from the handle of the delivery system, such that the handle may be used to advance the outer shaft 10, inner shaft 12, and occluder 200 through the lumen of the delivery sheath to the atrial septum.

Upon exposure from the delivery sheath, the distal region of outer shaft 10 and, accordingly, proximal end 210 coupled thereto may be moved towards the distal region of inner shaft 12 and, accordingly, distal end 212 and/or the distal region of inner shaft 12 and, accordingly, distal end 212 coupled thereto may be moved towards the distal region of outer shaft 10 and, accordingly, proximal end 210 coupled thereto to thereby transition proximal portion 202 of occluder 200 towards its expanded deployed state within the right atrium and transition distal portion 204 of occluder 200 towards its expanded deployed state within the left atrium while central portion 201 is positioned across the atrial septal defect. As proximal portion 202 and distal portion 204 of occluder 200 transition from the collapsed delivery state to the expanded deployed state, proximal portion 202 and distal portion 204 form disk-like structures that sandwich the atrial septal tissue therebetween.

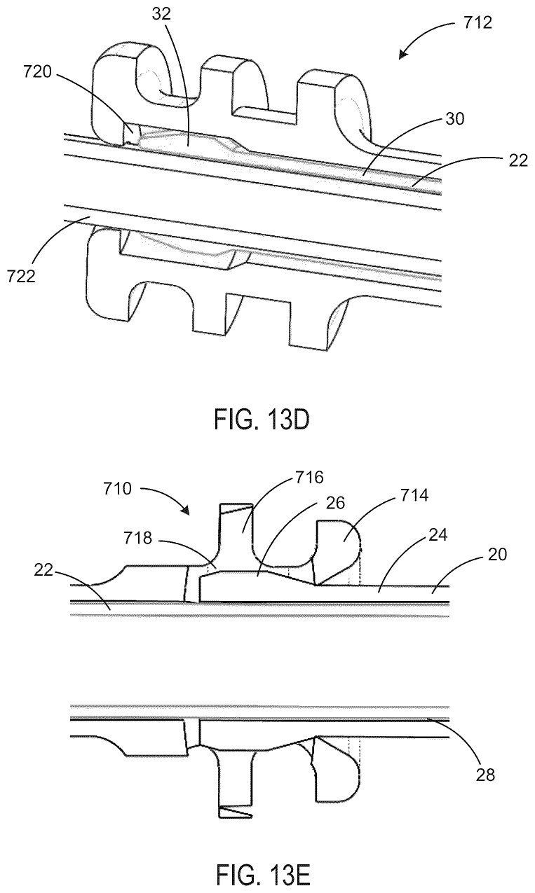

As described above, outer shaft 10 may be moved relative to inner shaft 12 until proximal end 210 locks to distal end 212, e.g., via a snap fit connection, and are separated by a predetermined distance D2, to thereby accommodate the system elasticity and dimensional tolerances. When proximal end 210 is locked to distal end 212, inner shaft 12 may be elastically elongated by the tensile load generated during locking of proximal end 210 to distal end 212. If proximal end 210 needs to be unlocked to distal end 212 for any reason, e.g. wrong size chosen, technical malfunction, etc., outer shaft 10 may be retracted relative to inner shaft 12 to disengage protrusion 220 from counterbore 222. To disengage inner shaft 12 from distal end 212, inner shaft 12 may be rotated relative to distal end 212, e.g., via actuation at the handle, to cause the threaded surface at the distal region of inner shaft 12 to decouple from the threaded surface of distal end 212 of occluder 200. As described above, inner shaft 12 may be elastically elongated by the tensile load generated during locking of proximal end 210 to distal end 212, such that, upon disengagement of inner shaft 12 from distal end 212 of occluder 200, inner shaft 12 may recoil, thereby ascertaining effective release of inner shaft 12 from distal end 212. To disengage outer shaft 10 from proximal end 210, outer shaft 10 may be rotated relative to proximal end 210, e.g., via actuation at the handle, to cause the threaded surface at the distal region of outer shaft 10 to decouple from the threaded surface of proximal end 210 of occluder 200. In some embodiments, the outer and inner shafts of the delivery system may be removably and reattachably coupled to the proximal and distal ends of the occluder via a threadless coupling mechanism, as described in further detail below with regard to FIGS. 13A to 15C.

Upon release from occluder 200, the delivery system may be removed from the patient's body, leaving occluder 200 implanted at the atrial septum. As will be understood by a person having ordinary skill in the art, prior to decoupling of occluder 200 from inner and outer shafts 12, 10 or after reconnecting occluder 200 to inner and outer shafts 12, 10 at the atrial septum, occluder 200 may be transitioned back to its collapsed delivery state, and optionally resheathed within the delivery sheath, to thereby permit the user to reposition/realign occluder 200 for proper implantation at the atrial septum and/or remove occluder 200 from the patient if necessary.

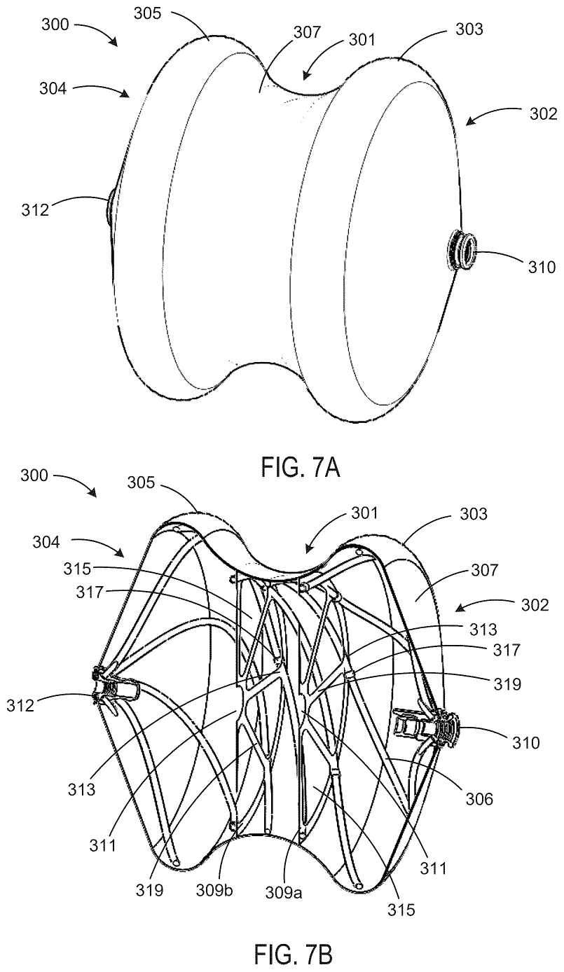

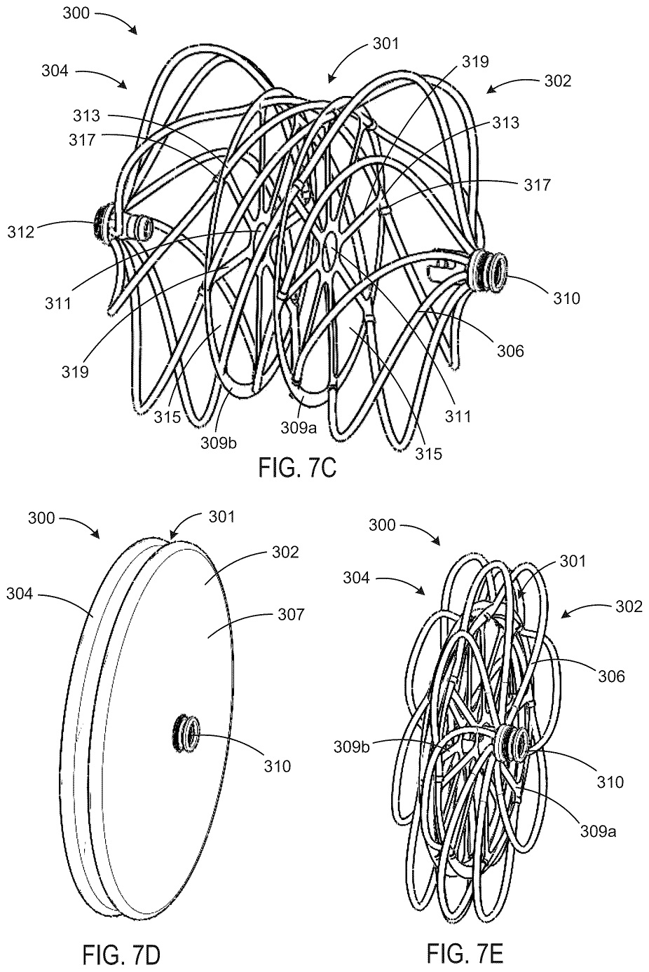

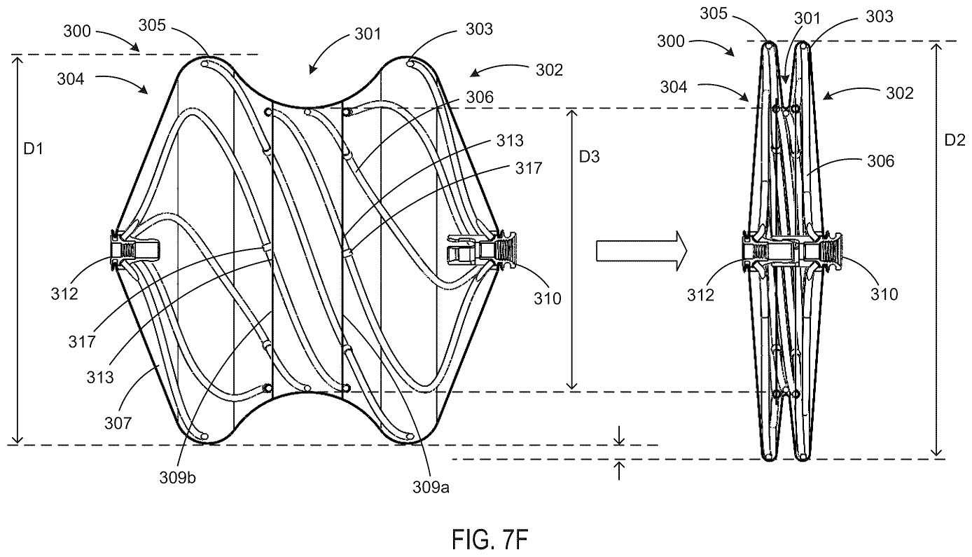

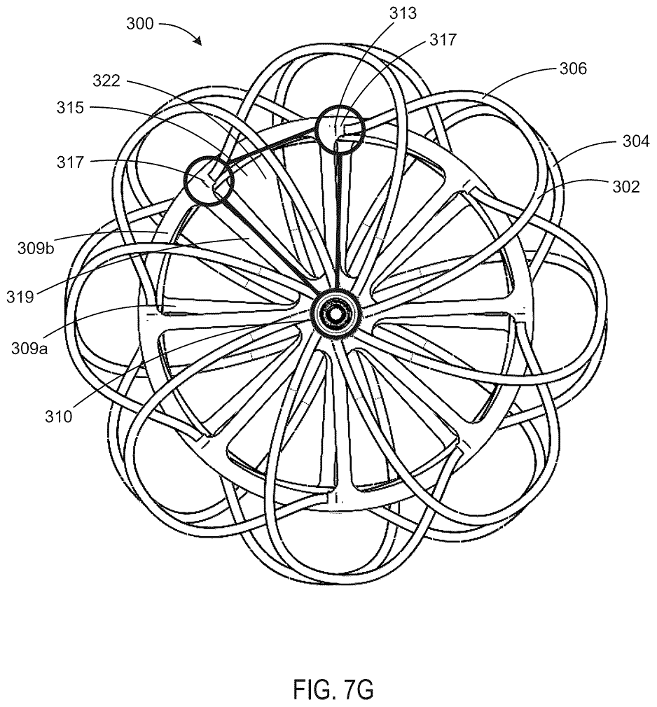

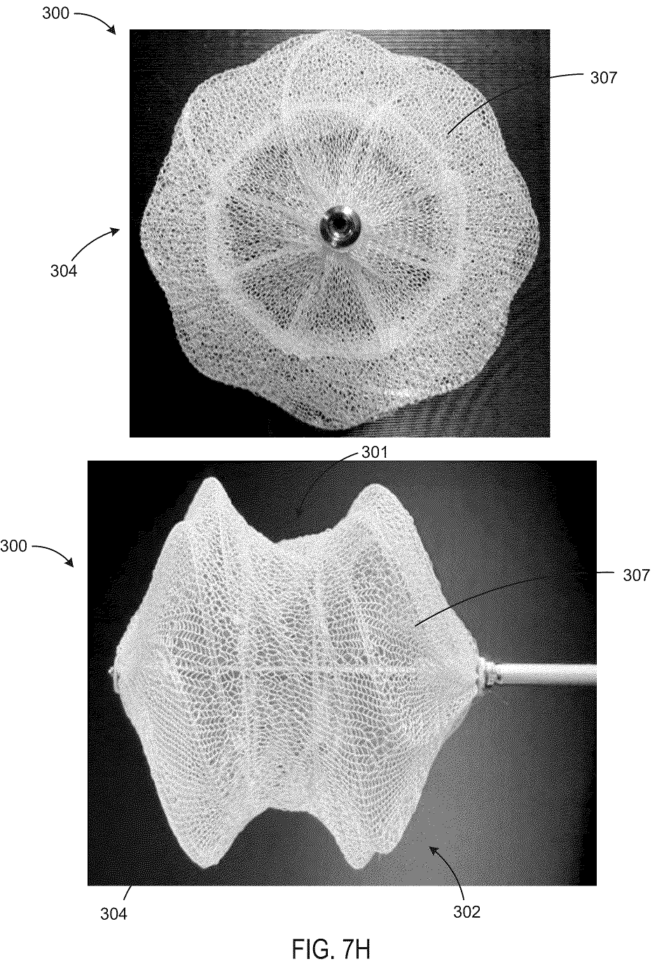

Referring now to FIGS. 7A to 7H, an alternative exemplary implantable occluder device is provided. The framework of implantable occluder device 300 may be constructed similar to the framework of implantable occluder device 200. For example, proximal end 310, distal end 312, and filaments 306 extending therebetween, correspond with proximal end 210, distal end 212, and filaments 206. Occluder 300 differs from occluder 200 in that, instead of two distinct biocompatible fabric patches 208a, 208b, biocompatible fabric patch 308 may be formed by proximal inner membrane 309a and distal inner membrane 309b (collectively referred to herein as inner membranes 309), and a biocompatible outer cover fabric 307 sized and shaped to be disposed over proximal portion 302, central portion 301, and distal portion 304 defined by filaments 306, and extending from proximal end 310 to distal end 312, as shown in FIGS. 7A to 7C. Outer cover fabric 307 may be coupled to the proximal and distal ends 310, 312 of occluder 300 via, e.g., mechanical crimping, chemical bonding, thermal bonding, and/or suturing. Preferably, outer cover fabric 307 may not be coupled to inner membranes 309 or to filaments 306, such that outer cover fabric 307 “floats” over inner membranes 309 and filaments 306 between proximal and distal ends 310, 312. Alternatively, in some embodiments, outer cover fabric 307 may be slidably coupled to individual points along filaments 306 (e.g., via suturing) to provide further structural support to filaments 306 as occluder 300 transitions between the collapsed delivery state and the expanded deployed state.

Inner membrane 309a may be disposed at the distal side of proximal portion 302 between proximal portion 302 and central portion 301, and inner membrane 309b may be disposed at the proximal side of distal portion 304 between distal portion 304 and central portion 301. Moreover, each of inner membranes 309 may comprise a wagon wheel shape. For example, as shown in FIGS. 7B and 7C, each of inner membranes 309 may include an inner ring defining aperture 411 at the center of the inner membrane, and an outer ring disposed circumferentially about and radially outward from the inner ring. The outer ring of the inner membrane may be coupled to the inner ring via a plurality of radially extending spokes 313, thereby defining a plurality of hollowed openings 315 between the plurality of radially extending spokes 313. Hollowed openings 315 may be configured to improve membrane crossability, e.g., if/when left atrium access is required, as described in further detail below with regard to FIG. 7G.

Additionally, each of inner membranes 309 may include a plurality of guiding holes 313 configured to guide passage of filaments 306 therethrough. For example, the plurality of guiding holes 313 may be circumferentially disposed along the outer ring of inner membranes 309. Accordingly, inner membranes 309 may provide the holding structure for the arrangement of filaments 306. Moreover, a plurality of radiopaque markers 317 may be disposed (e.g., sewn) on inner membranes 309, e.g., at guiding holes 313, and/or on filaments 306, e.g., adjacent to guiding holes 313. For example, radiopaque markers 317 may be sewn on the proximal side of the inner membrane, the distal side of the inner membrane, or both (e.g., in an alternating manner along the circumference of the inner membrane).

Outer cover fabric 307 may be a continuous and compliant member formed by, e.g., thin/ultra-thin weaved tubing, a knitted fabric (as shown in FIG. 7H), and/or electro-spun polymer such as polytetrafluoroethylene (PTFE), polyethylene terephthalate (PET), or polyurethane (PU), etc. For example, the knitted fabric may be heat set to provide more accurate outer profile matching with the framework of occluder 300. Outer cover fabric 307 may be configured to encapsulate filaments 306 during their degradation process to thereby prevent potential break off particles from embolizing, and may further be configured to promote cell growth (e.g., tissue overgrowth) upon implantation of occluder 300. FIGS. 7A to 7C illustrate occluder 300 in a partially expanded state with outer cover fabric 307 removed from FIG. 7C for brevity, and FIGS. 7D and 7E illustrate occluder 300 in its fully expanded deployed state with outer cover fabric 307 removed from FIG. 7E for brevity.

FIG. 7F illustrates occluder 300 as it transitions from the partially expanded state to the fully expanded deployed state where distal end 310 is removably coupled to proximal end 312, e.g., via a snap fit connection, as described above. As shown in FIG. 7F, outer edges 303, 305 of proximal and distal portions 302, 304, respectively, have a diameter D1 in the partially expanded state, and expands to diameter D2 in the fully expanded deployed state, while central portion 301 preferably has a constant diameter D3 in both the partially and fully expanded deployed states. As shown in FIG. 7G, when occluder 300 is in the fully expanded deployed state, radiopaque markers 317 and proximal end 310 and/or distal end 312 (not shown) may be visualized, e.g., under fluoroscopy, to identify the position of plurality of hollowed openings 315 (e.g., recross zones 322), which may advantageously be used as a triangular landmark for improving membrane crossability (e.g., recross) via occluder 300 when occluder 300 is implanted at the atrial septum and access to the left atrium from the right atrium is necessary.

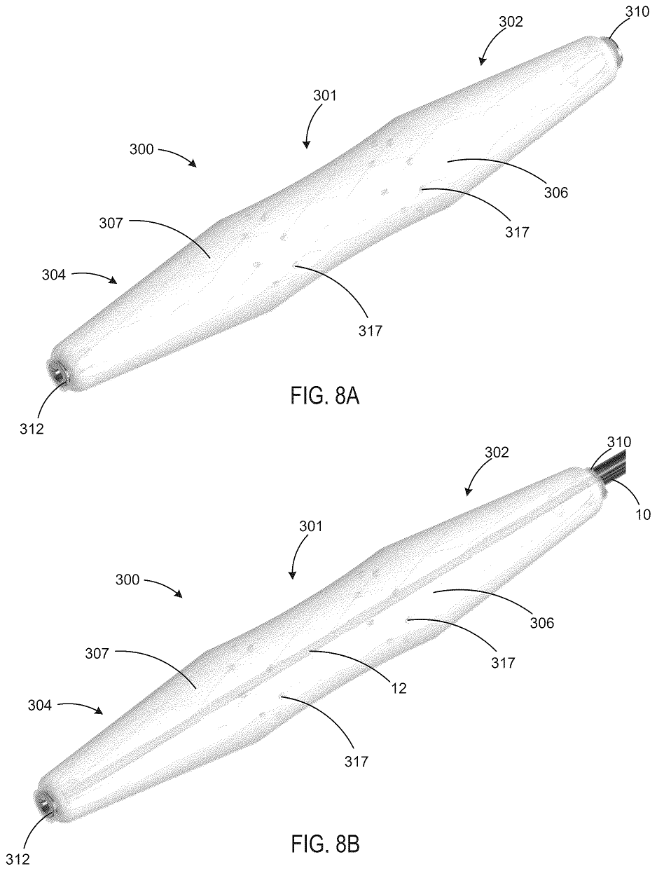

Referring now to FIG. 8A to 8E, delivery and implantation of occluder 300 is provided. As described above, occluder 300 may first be elongated by moving proximal end 312 and distal end 310 away from each other, thereby causing proximal portion 302, central portion 301, and distal portion 304 of occluder 300 to be collapsed radially inward towards the longitudinal axis of occluder 300, as shown in FIG. 8A. As shown in FIG. 8B, the threaded engagement portion at the distal end of inner shaft 12 may be removably and reattachably coupled to distal end 312 of occluder 300, e.g., by rotating distal end 312 relative to inner shaft 12, and the threaded engagement portion at the distal end of outer shaft 10 may be removably and reattachably coupled to proximal end 310 of occluder 300, e.g., by rotating proximal end 310 relative to outer shaft 10, as described above. In some embodiments, after distal end 312 is rotatably coupled to inner shaft 12 (e.g., in a first direction), rotation of proximal end 310 relative to outer shaft 10 (e.g., in a second direction opposite to the first direction) may also cause rotation of distal end 312 relative to inner shaft 12 in the second direction. Accordingly, the longitudinal length of the threaded surface of the engagement portion at the distal end of inner shaft 12 may be longer than the longitudinal length of the threaded surface of the engagement portion at the distal end of outer shaft 10, such that after distal end 312 is rotatably engaged with inner shaft 12, proximal end 310 may be rotatably coupled to outer shaft 10 without completely decoupling distal end 312 from inner shaft 12.

As described above, a delivery sheath may be advanced (e.g., over a guidewire) to the atrial septal defect, and occluder 300 may be advanced through the delivery sheath in its fully collapsed delivery state to the atrial septum, such that upon exposure from the distal end of the delivery sheath, distal portion 304 may be disposed within the left atrium, central portion 301 may be disposed through the atrial septal defect, and proximal portion 302 may be disposed within the right atrium. Next, occluder 300 may be transitioned towards its partially expanded deployed state, as shown in FIG. 8C, by actively moving proximal end 312 and distal end 310 toward each other via relative movement between inner shaft 12 and outer shaft 10, e.g., via one or more actuators at the handle of the delivery system. As shown in FIG. 8D, occluder 300 may be transitioned to its fully expanded deployed state by further moving proximal end 312 and distal end 310 toward each other via relative movement between inner shaft 12 and outer shaft 10 until proximal end 312 releasably engages with distal end 310. In the fully expanded deployed state, proximal portion 302 and distal portion 304 form disk-like structures that sandwich the atrial septal tissue therebetween.

As described above, occluder 300 may be re-collapsed (e.g., via relative movement between inner shaft 12 and outer shaft 10 and, accordingly, proximal end 312 and distal end 310) for repositioning at the atrial septum and/or removal. Once occluder 300 has been properly positioned at the atrial septal defect, outer shaft 10 may be decoupled from proximal end 310 (e.g., via rotation of proximal end 310 relative to outer shaft 10 in the first direction), and inner shaft 12 may be decoupled from distal end 312 (e.g., via rotation of distal end 312 relative to inner shaft 12 in the second direction), such that occluder 300 remains implanted at the atrial septum, as shown in FIG. 8E. Unlike other ASD occluder delivery systems, the delivery systems described herein may advantageously subsequently be reattached to the occluder, as necessary (e.g., for repositioning and/or removal), by rotatably re-coupling inner shaft 12 to distal end 312 and/or rotatably re-coupling outer shaft 10 to proximal end 310 in situ.

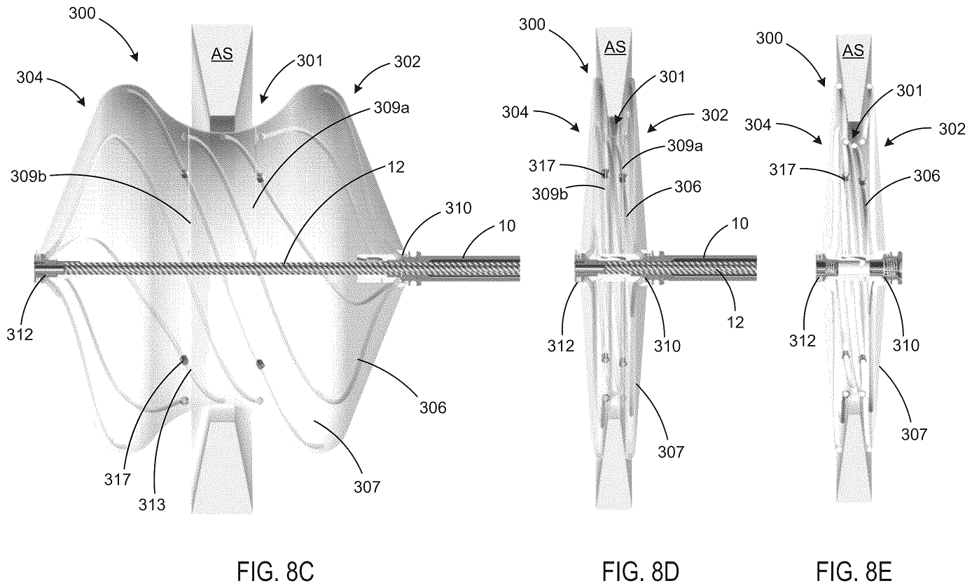

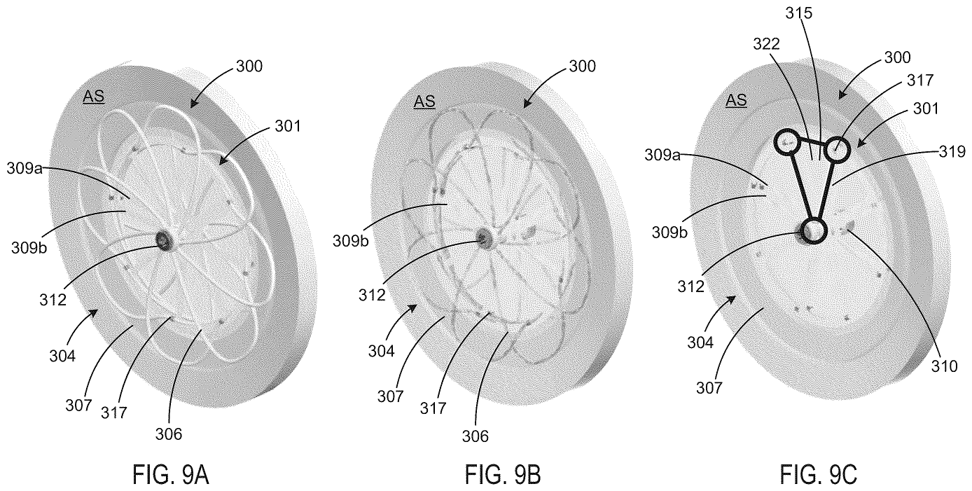

FIGS. 9A to 9D illustrate the degradation process of occluder 300 at various time periods post-implantation at atrial septum AS. As described above, the plurality of filaments 306 are biodegradable and, thus, degrade/resorb over time in the patient's body until only outer cover fabric 307, inner membranes 309, proximal end 310, distal end 312, and radiopaque markers 317 remain implanted at the atrial septum. For example, FIG. 9A illustrates occluder 300 implanted at atrial septum AS just after release from the delivery system, whereby filaments 306 are completely intact. FIG. 9B illustrates occluder 300 implanted at atrial septum AS during an interim degradation period post-implantation (e.g., 6-9 months post-implantation), whereby filaments 306 are partially degraded/resorbed. Moreover, filaments 306 may be fully degraded (e.g., complete resorption) within, e.g., 24 months, as shown in FIGS. 9C and 9D, whereby only outer cover fabric 307, proximal inner membrane 309a, distal inner membrane 309b, proximal end 310, distal end 312, and radiopaque markers 317 remain implanted at atrial septum AS. Alternatively, in some embodiments, the outer cover fabric and the proximal and distal inner membranes also may be formed of a bioresorbable material, preferably having a slower degradation/resorption rate than the plurality of filaments to thereby provide more time for tissue overgrowth to secure the non-bioresorbable components of the occluder device at the atrial septum. In such an embodiment, after complete degradation of the bioresorbable components of the occluder device at the atrial septum, only the proximal and distal ends of the occluder device and radiopaque markers will remain implanted at the atrial septum, e.g., secured thereto via tissue overgrowth.

Additionally, post-implantation, tissue ingrowth on occluder 300, i.e., endothelialization, further secures occluder 300 at atrial septum AS. As described above, when occluder 300 is implanted at the atrial septal defect, radiopaque markers 317 and proximal end 310 and/or distal end 312 may be visualized, e.g., under fluoroscopy, to identify the position of one or more recross zones 322 (e.g., hollowed openings 315), as shown in FIG. 9C, which may advantageously be used for recrossing the atrial septal defect, as described above, thereby improving membrane crossability. Accordingly, access to the left atrium from the right atrium may be achieved by advancing a medical device through recross zone 322 and, accordingly, through outer cover fabric 307. The crossability through the atrial septum post-implantation of the occluder designs described herein allows for further interventional techniques (e.g., mitral valve replacement, mitral valve repair, clipping the mitral valve leaflets (e.g., MitraClip from Abbott Laboratories), intracardiac ablation, left atrial appendage (LAA) occlusion devices, etc.). Conventional atrial septal occluders can block crossability through the atrial septum and, thus, these designs provide significant benefits.

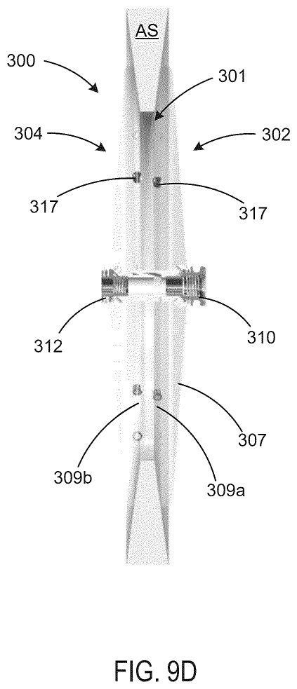

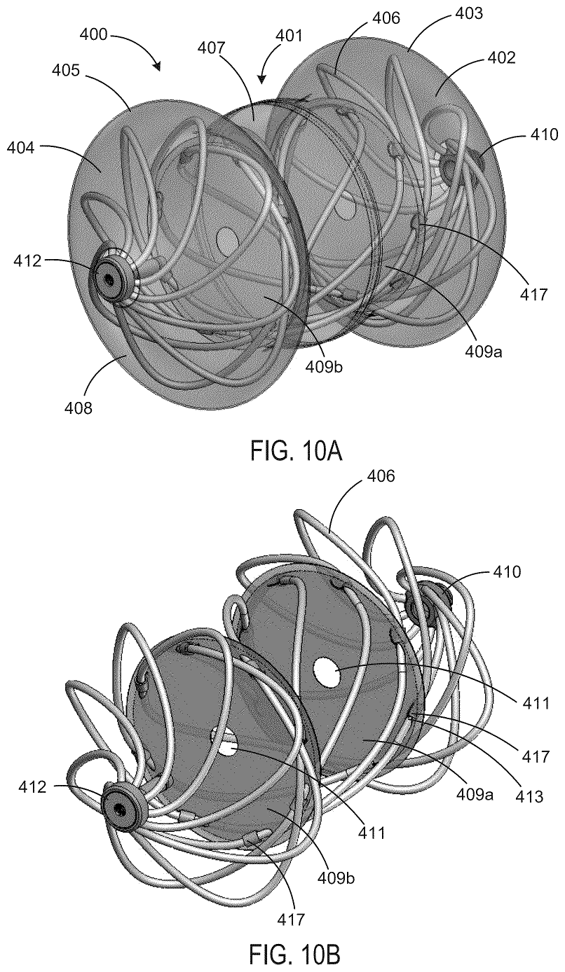

Referring now to FIGS. 10A and 10B, an alternative exemplary implantable occluder device is provided. Implantable occluder device 400 may be constructed similar to implantable occluder device 300. For example, the framework of occluder 400 includes proximal end 410, distal end 412, a plurality of filaments 406 extending therebetween, and a plurality of radiopaque markers 417, which correspond with proximal end 310, distal end 312, filaments 306, and radiopaque markers 317. Additionally, occluder 400 may further include biocompatible fabric patch 408 formed by proximal inner membrane 409a and distal inner membrane 409b (collectively referred to herein as inner membranes 409), and a compliant outer cover fabric 407 coupled to and extending between proximal end 410 and distal end 412 and sized and shaped to encapsulate proximal portion 402, central portion 401, and distal portion 404, as shown in FIG. 10A, which correspond with biocompatible fabric patch 308. Patch 408 differs from patch 308 in that, instead of wagon wheel shaped inner membranes 309, each of inner membranes 409 may comprise a full disk shape, as shown in FIG. 10B. For example, each of inner membranes 409 may include a thick ring portion defining aperture 411 sized and shaped to receive at least inner shaft 12 therethrough. In addition, the thick ring portion of inner membranes 409 further may include a plurality of guiding holes 413 configured to guide passage of filaments 406 therethrough, as shown in FIG. 10B.

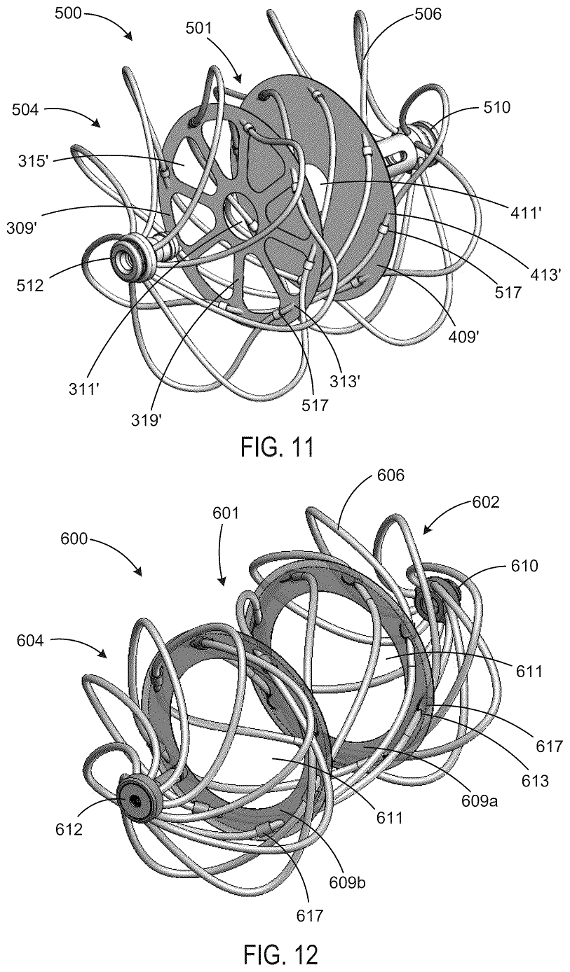

Referring now to FIG. 11, another alternative exemplary implantable occluder device is provided. Implantable occluder device 500 may be constructed similar to implantable occluder device 300. For example, the framework of occluder 500 includes proximal end 510, distal end 512, a plurality of filaments 506 extending therebetween, and a plurality of radiopaque markers 517, which correspond with proximal end 310, distal end 312, filaments 306, and radiopaque markers 317. Additionally, occluder 500 may further include a biocompatible fabric patch formed by a proximal inner membrane and a distal inner membrane, and a continuous and compliant outer cover fabric (not shown) coupled to and extending between proximal end 510 and distal end 512 and sized and shaped to encapsulate proximal portion 502, central portion 501, and distal portion 504, which correspond with biocompatible fabric patch 308. The biocompatible fabric patch of occluder 500 differs from patch 308 of occluder 300 in that, instead of two wagon wheel shaped inner membranes 309, the biocompatible fabric patch of occluder 500 may include a single wagon wheel shaped inner membrane (e.g., distal inner membrane 309′, which may be constructed similar to inner membrane 309 with similar components having like-prime reference numerals), and proximal full disk shaped inner membrane 409′, which may be constructed similar to inner membrane 409 with similar components having like-prime reference numerals. Alternatively, inner membrane 309′ may be the proximal inner member and inner membrane 409′ may be the distal inner membrane of occluder 500.

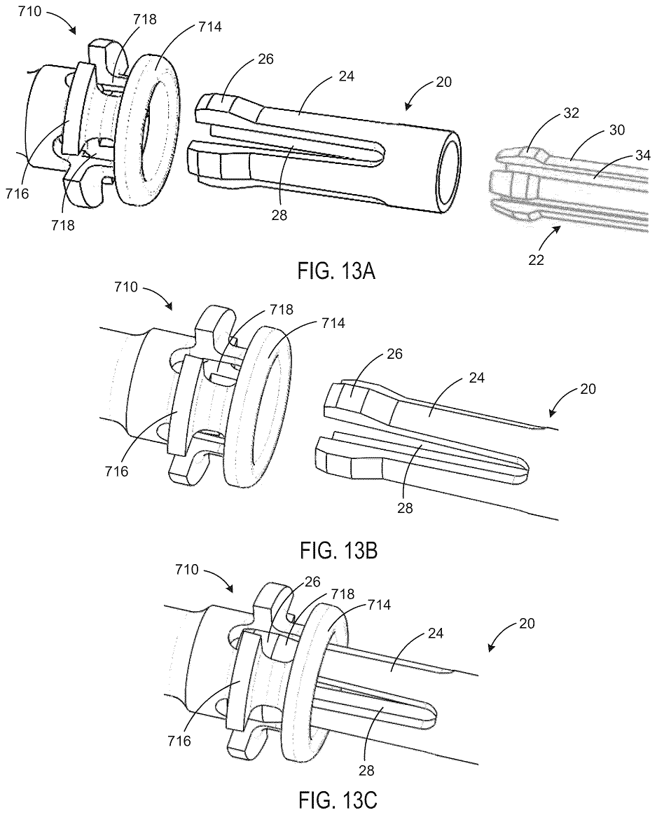

Referring now to FIG. 12, another alternative exemplary implantable occluder device is provided. Implantable occluder device 600 may be constructed similar to implantable occluder device 300. For example, the framework of occluder 600 includes proximal end 610, distal end 612, a plurality of filaments 606 extending therebetween, and a plurality of radiopaque markers 617, which correspond with proximal end 310, distal end 312, filaments 306, and radiopaque markers 317. Additionally, occluder 600 may further include a biocompatible fabric patch formed by proximal inner membrane 609a and distal inner membrane 609b (collectively referred to herein as inner membranes 609), and a continuous and compliant outer cover fabric (not shown) coupled to and extending between proximal end 610 and distal end 612 and sized and shaped to encapsulate proximal portion 602, central portion 601, and distal portion 604, which correspond with biocompatible fabric patch 308. The biocompatible fabric patch of occluder 600 differs from patch 308 of occluder 300 in that, instead of wagon wheel shaped inner membranes 309, each of inner membranes 609 may comprise a ring shape, as shown in FIG. 12. For example, each of inner membranes 609 may include a thin ring portion defining aperture 611, which may be magnitudes larger than aperture 411 of inner membranes, 409a, 409b. Aperture 611 may be sized and shaped to improve membrane crossability, e.g., if/when left atrium access is required, as described above. In addition, the thin ring portion of inner membranes 609 further may include a plurality of guiding holes 613 configured to guide passage of filaments 606 therethrough, as shown in FIG. 12. As will be understood by a person having ordinary skill in the art, the occluders described herein may include any combination of inner membranes (e.g., inner membranes 309, 409, 609).

Referring now to FIGS. 13A to 13E, an alternative exemplary coupling mechanism for removably and reattachably coupling the outer and inner shafts of an ASD occluder delivery system to the proximal and distal ends of an implantable occluder device is provided. For example, instead of the threaded proximal and distal ends described above, the implantable occluder devices described herein may include proximal end 710 configured for removably and reattachably engaging with the engagement portion at the distal region of the outer shaft of the delivery system (e.g., outer shaft 20), and/or distal end 712 configured for removably and reattachably engaging with the engagement portion at the distal region of the inner shaft of the delivery system (e.g., inner shaft 22), as shown in FIG. 13A. Proximal end 710 and distal end 712 may be constructed similar to the proximal and distal ends described above in that proximal end 710 and distal end 712 are configured to be releasably coupled together to thereby lock the occluder device in its fully expanded deployed state, as described above (e.g., by releasably locking the protrusion of distal end 712 within the counterbore of proximal end 710, as described above).

Unlike the threaded proximal ends described above, proximal end 710 may include proximal stopper 714 extending radially outwardly and circumferentially about the outer surface of proximal end 710, and a plurality of radially outwardly extending distal protrusions 716 disposed circumferentially along the outer surface of proximal end 710. As shown in FIG. 13B, the plurality of distal protrusions 716 may be separated by a plurality of openings 718 extending through the outer surface of proximal end 710 in communication with the central lumen of proximal end 710. At least the proximal region of the central lumen of proximal end 710 may be sized and shaped to receive the engagement portion at the distal region of outer shaft 20 for removably coupling proximal end 710 to outer shaft 20. For example, as shown in FIG. 13B, the engagement portion at the distal region of outer shaft 20 may be formed of a plurality of longitudinally extending arms 24 joined together at the proximal end of arms 24 and extending longitudinally towards the distal end of outer shaft 20, wherein each adjacent arm 24 is separated by a longitudinally extending slit 28. Arms 24 are flexible such that they may bend radially inward upon application of force, but are biased towards the non-bended state. As will be understood by a person having ordinary skill in the art, while FIG. 13A illustrates outer shaft 20 having four arms 24, outer shaft 20 may have more or less than four arms 24. The portion of outer shaft 20 proximal to the engagement portion of outer shaft 20 is not shown in FIG. 13A for brevity.

Additionally, the distal portion of arms 24 may include a plug (e.g., protrusions 26) extending radially outward from the outer surface of the engagement portion of outer shaft 20. Each protrusion 26 may be sized and shaped to at least partially extend through openings 718 of proximal end 710. Accordingly, to removably and reattachably couple outer shaft 20 to proximal end 710, the engagement portion of outer shaft 20 may be inserted into the lumen of proximal end 710, such that stopper 714 causes arms 24 to bend radially inward (e.g., via contact between stopper 714 and protrusions 26 as outer shaft 20 is inserted into the lumen of proximal end 710). When protrusions 26 are aligned with openings 718, protrusions 26 are no longer constrained within the lumen of proximal end 710 and, thus, arms 24 may return to their non-bended state, such that protrusions 26 at least partially extend through openings 718 to thereby releasably secure outer shaft 20 to proximal end 710, as shown in FIG. 13C. For example, protrusions 26 may be tightly disposed within openings 718, such that the distal end of openings 718 prevents distal movement of protrusions 26 and, accordingly, outer shaft 20 relative to proximal end 710, and stopper 714 prevents proximal movement of protrusions 26 and, accordingly, outer shaft 20 relative to proximal end 710. Moreover, adjacent protrusions 716 may prevent rotation of protrusions 26 and, accordingly, outer shaft 20 relative to proximal end 710. In addition, as shown in FIG. 13A, outer shaft 20 may comprise a lumen sized and shaped to slidably receive inner shaft 22 therethrough.

As shown in FIG. 13A, the engagement portion at the distal region of inner shaft 22 similarly may be formed of a plurality of longitudinally extending arms 30 joined together at the proximal end of arms 30 and extending longitudinally towards the distal end of inner shaft 22, wherein each adjacent arm 30 is separated by a longitudinally extending slit 34. Arms 30 are flexible such that they may bend radially inward upon application of force, but are biased towards the non-bended state. Additionally, the distal portion of arms 30 may include a plug (e.g., protrusions 32) extending radially outward from the outer surface of the engagement portion of inner shaft 22 for removable and reattachable engagement with distal end 712.

As shown in FIG. 13D, the lumen of distal end 712 may comprise an undercut (e.g., counterbore 720) extending circumferentially along the inner surface of a distal portion of the lumen. Counterbore 720 may be sized and shaped to releasably and securely receive protrusions 32 of inner shaft 22, e.g., via a snap fit connection. Thus, counterbore 720 may have a geometry that corresponds with the geometry of protrusions 32, such that when protrusions 32 are disposed within counterbore 720, the engagement portion of inner shaft 22 is releasably secured to distal end 712. In some embodiments, counterbore 720 may comprise one or more openings configured to allow for transfer of torque/rotation of inner shaft 22 relative to distal end 712. For example, counterbore 720 may comprise a plurality of discrete receptacles disposed within and circumferentially along the lumen of distal end 712, each discrete receptacle sized and shaped to releasably and securely receive protrusion 32 therein, e.g., in a manner that prevent rotation of inner shaft 22 relative to distal end 712. As shown in FIG. 13D, the proximal side of protrusions 32 and proximal end of counterbore 720 may comprise corresponding tapered profiles configured to facilitate release of the engagement portion of inner shaft 22 from distal end 712.

Accordingly, to removably and reattachably couple inner shaft 22 to distal end 712 (e.g., after the engagement portion of outer shaft 20 has been removably coupled to proximal end 710), inner shaft 22 may be advanced through the lumen of outer shaft 20 to thereby insert the engagement portion of inner shaft 22 into the lumen of distal end 712, such that the inner wall of the lumen of distal end 712 causes arms 30 to bend radially inward as inner shaft 22 is inserted into the lumen of distal end 712 (e.g., via contact between the inner wall of the lumen of distal end 712 and protrusions 32 as inner shaft 22 is inserted into the lumen of distal end 712). When protrusions 32 are aligned with counterbore 720, protrusions 32 are no longer constrained within the lumen of distal end 712 and, thus, arms 30 may return to their non-bended state, such that protrusions 32 at least partially extend into counterbore 720 to thereby releasably secure inner shaft 22 to distal end 712, as shown in FIG. 13D.

For example, protrusions 32 may be tightly disposed within counterbore 720, such that the distal end of counterbore 720 prevents distal movement of protrusions 32 and, accordingly, inner shaft 22 relative to distal end 712, and the proximal end of counterbore 720 prevents proximal movement of protrusions 32 and, accordingly, inner shaft 22 relative to distal end 712. Additionally, as shown in FIG. 13D, a core (e.g., tube 722) may be inserted through the lumen of inner shaft 22 to thereby prevent arms 30 of inner shaft 22 from bending radially inward, thereby locking protrusions 32 of inner shaft 22 within counterbore 720 of distal end 712. For example, tube 722 may comprise guidewire tube having a lumen sized and shaped to slidably receive a guidewire therethrough. Alternatively, in some embodiments, instead of tube 722, the guidewire itself may function to prevent arms 30 of inner shaft 22 from bending radially inward when the guidewire is disposed within the lumen of inner shaft 22.

Moreover, as shown in FIG. 13E, when inner shaft 22 is disposed within the lumen of outer shaft 20, inner shaft 22 may prevent arms 24 of outer shaft 20 from bending radially inward, thereby locking protrusions 26 of outer shaft 20 within openings 718 of proximal end 710. Accordingly, inner shaft 22 may be retracted within the lumen of outer shaft 20 such that the engagement portion of inner shaft 22 is proximal to the engagement portion of outer shaft 20 prior to removably and reattachably coupling the engagement portion of outer shaft 20 to proximal end 710. Accordingly, to decouple inner shaft 22 from distal end 712, tube 722 may be retracted within inner shaft 22 to permit arms 30 to bend radially inward upon proximal movement of inner shaft 22 relative to distal end 712 (e.g., via contact between the tapered proximal end of counterbore 720 and the tapered proximal side of protrusions 32 as inner shaft 22 moved proximally relative to distal end 712). As will be understood by a person having ordinary skill in the art, the implantable occluder devices described herein may include proximal end 710 (and, accordingly the delivery system may include outer shaft 20), and any of the threaded distal ends described above (and, according the delivery system may include threaded inner shaft 12).

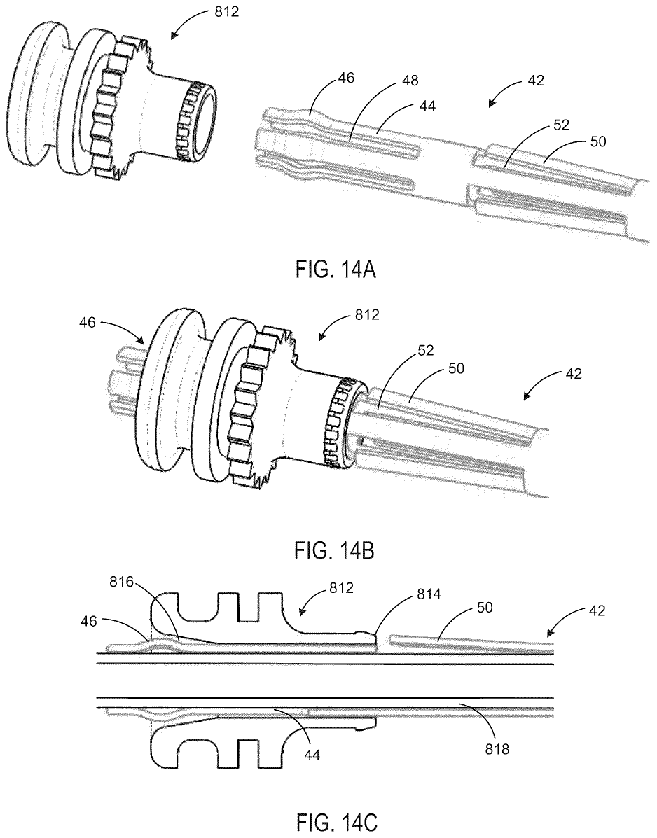

Referring now to FIGS. 14A to 14C, an alternative exemplary coupling mechanism for removably and reattachably coupling the inner shaft of an ASD occluder delivery system to the distal end of an implantable occluder device is provided. Inner shaft 42 may be constructed similar to inner shaft 22 of FIG. 13A. For example, the engagement portion at the distal region of inner shaft 42 similarly may be formed of a plurality of longitudinally extending arms 44 joined together at the proximal end of arms 44 and extending longitudinally towards the distal end of inner shaft 42, wherein each adjacent arm 44 is separated by a longitudinally extending slit 48. Arms 44 are flexible such that they may bend radially inward upon application of force, but are biased towards the non-bended state. Additionally, the distal portion of arms 44 may include protrusions 46 extending radially outward from the outer surface of the engagement portion of inner shaft 42 for removable and reattachable engagement with distal end 812. Moreover, inner shaft 22 similarly may comprise a lumen sized and shaped to receive a core (e.g., tube 818, which may comprise a guidewire tube having a lumen sized and shaped to slidably receive a guidewire therethrough).

Unlike inner shaft 22, the engagement portion of inner shaft 42 further may include a plurality of expandable flaps 50 proximal to arms 44 and configured to transition between a collapsed position where flaps 50 are disposed within a plurality of corresponding receptacles 52, and an expanded position where flaps 50 extend radially outward in the distal direction from the outer surface of the engagement portion of inner shaft 42 to thereby prevent distal movement of inner shaft 42 relative to distal end 812 when inner shaft 42 is removably and reattachably coupled to distal end 812, as described in further detail below. The proximal end of flaps 50 may be pivotally/hingedly coupled to inner shaft 42 and flaps 50 may be biased toward the expanded position, such that the free distal ends of flaps 50 may be configured to transition from the expanded position to the collapsed position via application of force. In some embodiments, flaps 50 may be formed as a cut out of inner shaft 42, the cut out defining the corresponding receptacles 52.

Distal end 812 may be constructed similar to distal end 712 of FIG. 13D in that distal end 812 may be coupled to the distal end of the occluder device and configured to be releasably coupled to any of the proximal ends described herein to thereby lock the occluder device in its fully expanded deployed state, as described above (e.g., by releasably locking the protrusion of distal end 812 within the counterbore of the proximal end, as described above). Moreover, distal end 812 similarly may comprise a lumen sized and shaped to receive the engagement portion of inner shaft 42 therethrough. Distal end 812 differs from distal end 712 in that instead of a counterbore that is fully disposed within the lumen of distal end 812, distal end 812 may include counterbore 816 extending circumferentially along the inner surface at the distal end of the lumen of distal end 812, as shown in FIG. 14C.

Counterbore 816 may be sized and shaped to releasably and securely receive at least a portion of protrusions 46 of inner shaft 42, e.g., via a snap fit connection. Thus, counterbore 816 may have a geometry that corresponds with the geometry of protrusions 46, such that when protrusions 46 are disposed within counterbore 816, the proximal end of counterbore 816 prevents proximal movement of protrusions 46 and, accordingly, inner shaft 42 relative to distal end 812. Alternatively, in some embodiments, counterbore 816 may comprise a plurality of discrete receptacles disposed within and circumferentially along the lumen of distal end 812, each discrete receptacle sized and shaped to releasably and securely receive protrusion 46 therein, e.g., in a manner that prevent rotation of inner shaft 42 relative to distal end 812. As shown in FIG. 14C, the proximal side of protrusions 46 and proximal end of counterbore 816 may comprise corresponding tapered profiles configured to facilitate release of the engagement portion of inner shaft 42 from distal end 812.

Accordingly, to removably and reattachably couple inner shaft 42 to distal end 812 (e.g., after the engagement portion of the outer shaft has been removably coupled to the proximal end), inner shaft 42 may be advanced through the lumen of the outer shaft to thereby insert the engagement portion of inner shaft 42 into the lumen of distal end 812, such that the inner wall of the lumen of distal end 812 causes arms 44 to bend radially inward as inner shaft 42 is inserted into the lumen of distal end 812 (e.g., via contact between the inner wall of the lumen of distal end 812 and protrusions 46 as inner shaft 42 is inserted into the lumen of distal end 812). When protrusions 46 are aligned with counterbore 46, protrusions 46 are no longer constrained within the lumen of distal end 812 and, thus, arms 44 may return to their non-bended state, such that protrusions 46 at least partially extend into counterbore 816 and the proximal end of counterbore 816 prevents proximal movement of protrusions 46 and, accordingly, inner shaft 42 relative to distal end 812. Moreover, as shown in FIGS. 14B and 14C, when protrusions 46 are disposed within counterbore 816 and flaps 50 are in their expanded position, proximal end 814 of distal end 812 prevents distal movement of flaps 50 and, accordingly, inner shaft 42 relative to distal end 812. Accordingly, the engagement between protrusions 46 and counterbore 816 and the engagement between flaps 50 and proximal end 814 of distal end 812 together releasably secure the engagement portion of inner shaft 42 to distal end 812.

Additionally, as shown in FIG. 14C, tube 818 may be inserted through the lumen of inner shaft 42 to thereby prevent arms 44 of inner shaft 42 from bending radially inward, thereby locking protrusions 46 of inner shaft 42 within counterbore 816 of distal end 812. Alternatively, in some embodiments, instead of tube 818, the guidewire itself may function to prevent arms 44 of inner shaft 42 from bending radially inward when the guidewire is disposed within the lumen of inner shaft 42. Accordingly, to decouple inner shaft 42 from distal end 812, tube 818 may be retracted within inner shaft 42 to permit arms 44 to bend radially inward upon proximal movement of inner shaft 42 relative to distal end 812 (e.g., via contact between the tapered proximal end of counterbore 816 and the tapered proximal side of protrusions 46 as inner shaft 42 moved proximally relative to distal end 812). As will be understood by a person having ordinary skill in the art, the implantable occluder devices described herein may include distal end 812 (and, accordingly the delivery system may include inner shaft 42), and any of the (threaded or threadless) proximal ends described above (and, according the delivery system may include a corresponding outer shaft).

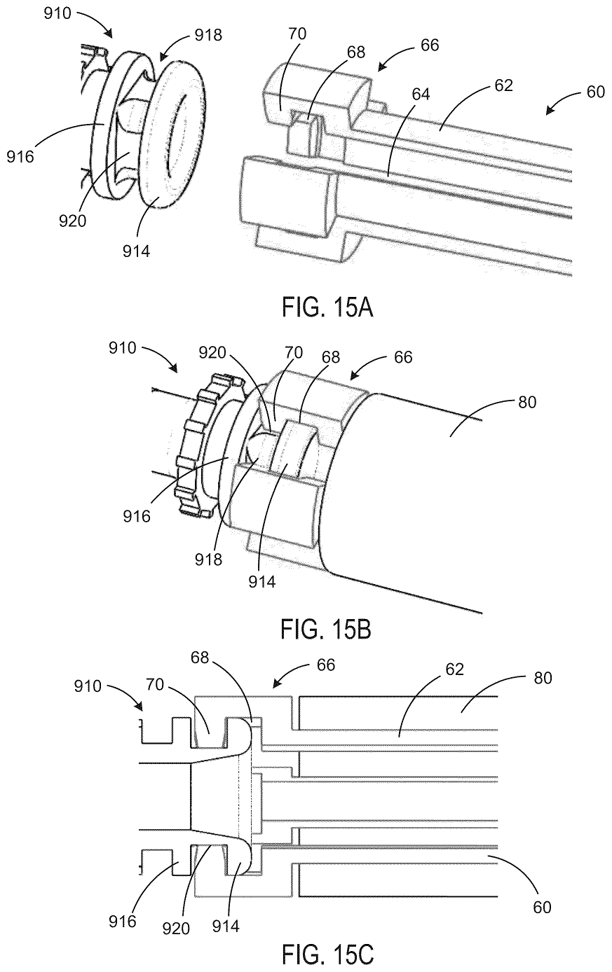

Referring now to FIGS. 15A to 15C, an alternative exemplary coupling mechanism for removably and reattachably coupling the outer shaft of an ASD occluder delivery system to the proximal end of an implantable occluder device is provided. Outer shaft 60 may be constructed similar to outer shaft 20 of FIG. 13A. For example, the engagement portion at the distal region of outer shaft 60 similarly may be formed of a plurality of longitudinally extending arms 62 joined together at the proximal end of arms 62 and extending longitudinally towards the distal end of outer shaft 60, wherein each adjacent arm 62 is separated by a longitudinally extending slit 64.

Outer shaft 60 differs from outer shaft 20 in that the distal portion of each arm 62 comprises a grabber (e.g., hook portion 66) extending radially outward from the outer surface of outer shaft 60 and defining teeth portion 70 and receptacle 68 sized and shaped to releasably engage with stopper 914 of proximal end 910 for removable and reattachable engagement with proximal end 912, as described in further detail below. Moreover, arms 62 may be configured to transition between a collapsed position where arms 62 extend parallel to the longitudinal axis of outer shaft 60, as shown in FIG. 15A, and an expanded position where arms 62 extend radially outward from the outer surface of the engagement portion of outer shaft 60. The proximal end of arms 62 (not shown) may be pivotally/hingedly coupled to outer shaft 60 and arms 62 may be biased toward the expanded position, such that the free distal ends of arms 62 may be configured to transition from the expanded position to the collapsed position via application of force (e.g., by sliding an external tube over outer shaft 60 towards hook portion 66, as described in further detail below).

Proximal end 910 may be constructed similar to the proximal end 710 of FIG. 13A in that proximal end 910 may be coupled to the proximal end of the occluder device and configured to be releasably coupled to any of the distal ends described herein to thereby lock the occluder device in its fully expanded deployed state, as described above (e.g., by releasably locking the protrusion of the distal end within the counterbore of proximal end 910, as described above). Moreover, as shown in FIG. 15A, proximal end 910 similarly may include proximal stopper 914 extending radially outwardly and circumferentially about the outer surface of proximal end 910 and a lumen sized and shaped to receive the inner shaft of the delivery system therethrough. Proximal end 910 differs from proximal end 710 in that proximal end 910 further may include distal stopper 916 disposed distal to proximal stopper 914 and defining gap 918 therebetween, as shown in FIG. 15A. Additionally, the outer surface of distal end 910 within gap 918 may comprise a plurality of discrete planar surfaces 920 disposed circumferentially about the longitudinal axis of distal end 910. For example, the number of planar surfaces 920 may correspond with the number of hook portions 66 of outer shaft 60.

Accordingly, to removably and reattachably couple outer shaft 60 to proximal end 910, arms 62 at the engagement portion of outer shaft 60 may be transitioned to their biased expanded position (e.g., by removing any constraints, such as jacket/sleeve from over arms 62), and advanced over the proximal portion of proximal end 910, such that teeth 70 of hook portions 70 are aligned with gap 918 and, particularly, with planar surfaces 920 within gap 918, and receptacles 68 are aligned with proximal stopper 914. An outer jacket/sleeve (e.g., tube 80) may then be advanced distally over arms 62 of outer shaft 60 until the distal end of tube 80 abuts the proximal end of hook portions 66, thereby causing arms 62 to bend radially inward toward the collapsed position, such that teeth 70 engages with planar surfaces 920 within gap 918 and proximal stopper 914 is releasably received within receptacles 68 to thereby removably and reattachably secure the engagement portion of outer shaft 60 to proximal end 910, as shown in FIGS. 15B and 15C. Accordingly, the proximal end of tube 80 may be operatively coupled to the handle of the delivery system. Moreover, the engagement between teeth 70 and planar surfaces 920 may advantageously allow for transmission of torque/rotation from outer shaft 60 to proximal end 910.

To decouple the engagement portion of outer shaft 60 from proximal end 910, tube 80 may be retracted relative to outer shaft 60 to thereby expose arms 62 from the distal end of tube 80 and permit arms 62 to return to their biased expanded positions whereby teeth 70 is disengaged from planar surfaces 920 and proximal stopper 914 is disengaged from receptacles 68. As will be understood by a person having ordinary skill in the art, the implantable occluder devices described herein may include proximal end 910 (and, accordingly the delivery system may include outer shaft 60), and any of the (threaded or threadless) distal ends described above (and, according the delivery system may include a corresponding inner shaft).

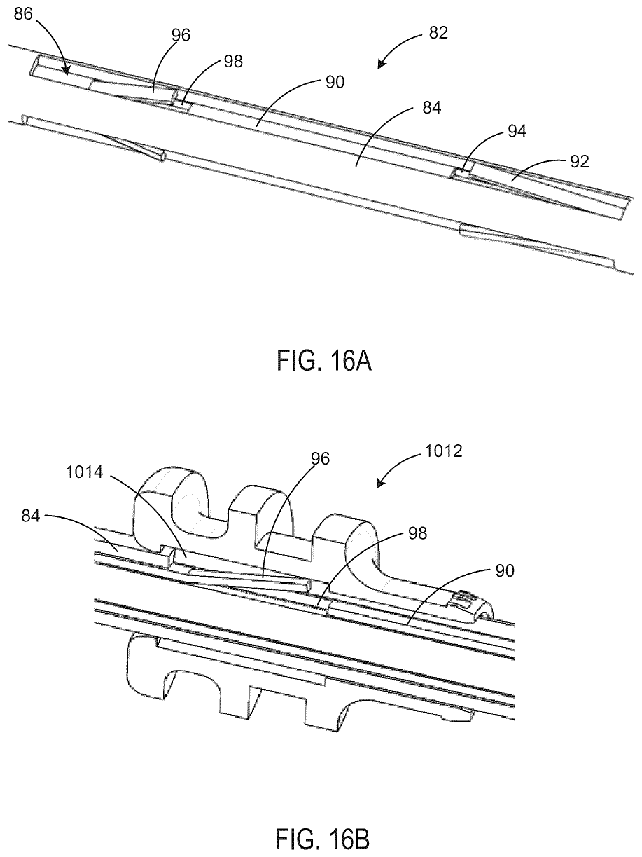

Referring now to FIGS. 16A and 16B, another alternative exemplary coupling mechanism for removably and reattachably coupling the inner shaft of an ASD occluder delivery system to the distal end of an implantable occluder device is provided. As shown in FIG. 16A. inner shaft 82 may include concentric outer tube 84 and concentric inner tube 90 slidably disposed within the lumen of outer tube 84. Accordingly, the proximal ends of outer tube 84 and/or inner tube 90 may be independently operatively coupled to the handle of the delivery system for actuating movement of outer tube 84 relative to inner tube 90. As shown in FIG. 16A, at the engagement portion at the distal region of inner shaft 82, outer tube 84 may comprise a plurality of longitudinally extending slits 86, each slit 86 sized and shaped to at least partially receive proximal flap 92 and distal flap 96 of inner tube 90 therethrough.

Particularly, at the engagement portion of inner shaft 82, inner tube 90 may include a plurality of expandable proximal flaps 92 disposed circumferentially along the outer surface of inner tube 90, and a plurality of expandable distal flaps 96 disposed circumferentially along the outer surface of inner tube 90 distal to proximal flaps 92. The number of proximal flaps 92 and distal flaps 96 may correspond with the number of slits 86 of outer tube 84. Proximal flaps 92 may be configured to transition between a collapsed position where proximal flaps 92 are disposed within a plurality of corresponding receptacles 94, and an expanded position where proximal flaps 92 extend radially outward in the distal direction from the outer surface of inner tube 90 through corresponding slits 86 to thereby prevent distal movement of inner shaft 82 relative to distal end 1012 beyond the position of proximal flaps 92. Moreover, the proximal end of proximal flaps 92 may be pivotally/hingedly coupled to inner tube 90 and proximal flaps 92 may be biased toward the expanded position, such that the free distal ends of proximal flaps 92 may be configured to transition from the expanded position to the collapsed position via application of force. In some embodiments, proximal flaps 92 may be formed as a cut out of inner tube 90, the cut out defining the corresponding receptacles 94.

Similarly, distal flaps 96 may be configured to transition between a collapsed position where distal flaps 96 are disposed within a plurality of corresponding receptacles 98, and an expanded position where distal flaps 96 extend radially outward in the proximal direction from the outer surface of inner tube 90 through corresponding slits 86 to thereby prevent proximal movement of inner shaft 82 relative to distal end 1012 when inner shaft 82 is removably and reattachably coupled to distal end 1012, as described in further detail below. Moreover, the distal end of distal flaps 96 may be pivotally/hingedly coupled to inner tube 90 and distal flaps 96 may be biased toward the expanded position, such that the free proximal ends of distal flaps 96 may be configured to transition from the expanded position to the collapsed position via application of force. In some embodiments, distal flaps 96 may be formed as a cut out of inner tube 90, the cut out defining the corresponding receptacles 98.

Accordingly, as outer tube 84 is slidably disposed over inner tube 90, movement of outer tube 84 distally relative to inner tube 90 may cause proximal flaps 92 to transition to its collapsed position within receptacle 94, e.g., when the proximal end of slits 86 is distal to proximal flaps 92, such that proximal flaps 92 are disposed within the lumen of outer tube 84. Similarly, movement of outer tube 84 proximally relative to inner tube 90 may cause distal flaps 96 to transition to its collapsed position within receptacle 98, e.g., when the distal end of slits 86 is proximal to distal flaps 96, such that distal flaps 96 are disposed within the lumen of outer tube 84.

Distal end 1012 may be constructed similar to distal end 712 of FIG. 13D in that distal end 1012 may be coupled to the distal end of the occluder device and configured to be releasably coupled to any of the proximal ends described herein to thereby lock the occluder device in its fully expanded deployed state, as described above (e.g., by releasably locking the protrusion of distal end 1012 within the counterbore of the proximal end, as described above). Moreover, distal end 1012 similarly may comprise a lumen sized and shaped to receive the engagement portion of inner shaft 82 therethrough, as well as counterbore 1014 extending circumferentially along the inner surface of the lumen of distal end 1012, as shown in FIG. 16B.

Counterbore 1014 may be sized and shaped to releasably and securely receive distal flaps 96 therein in the expanded position, e.g., via a snap fit connection. Thus, counterbore 1014 may have a geometry that corresponds with the geometry of distal flaps 96, such that when distal flaps 96 are disposed within counterbore 1014 in the expanded position, the proximal end of counterbore 1014 prevents proximal movement of distal flaps 96 and, accordingly, inner tube 90 of inner shaft 82 relative to distal end 1012. Alternatively, in some embodiments, counterbore 1014 may comprise a plurality of discrete receptacles disposed within and circumferentially along the lumen of distal end 1012, each discrete receptacle sized and shaped to releasably and securely receive distal flaps 96 therein, e.g., in a manner that prevent rotation of inner tube 90 of inner shaft 82 relative to distal end 1012.

Accordingly, to removably and reattachably couple inner shaft 82 to distal end 1012 (e.g., after the engagement portion of the outer shaft has been removably coupled to the proximal end), outer tube 84 may first be moved distally relative to inner tube 90 to collapse proximal flaps 92 within the lumen of outer tube 84, to thereby prevent interference between proximal flaps 92 and the outer shaft as inner shaft 82 is inserted through the lumen of the outer shaft. Outer and inner tubes 84, 90 may then be advanced together distally through the lumen of the outer shaft, and then through the lumen of distal end 1012. As inner shaft 82 is advanced distally through the lumen of the outer shaft, contact between the inner wall of the lumen of the outer shaft and distal flaps 96 causes distal flaps 96 to transition towards their collapsed position within the lumen of the outer shaft, thereby constraining distal flaps 96 within the outer shaft. Similarly, as inner shaft 82 is advanced distally through the lumen of distal end 1012, contact between the inner wall of the lumen of distal end 1012 and distal flaps 96 causes distal flaps 96 to transition towards their collapsed position within the lumen of distal end 1012, thereby constraining distal flaps 96 within distal end 1012.

When distal flaps 96 are aligned with counterbore 1014, distal flaps 96 are no longer constrained within the lumen of distal end 1012 and, thus, distal flaps 96 may return to their biased expanded position, such that distal flaps 96 at least partially extend into counterbore 1014 and the proximal end of counterbore 1014 prevents proximal movement of distal flaps 96 and, accordingly, inner tube 90 of inner shaft 82 relative to distal end 1012. Moreover, when distal flaps 96 are disposed within counterbore 1014 and proximal flaps 92 are in their expanded position proximal to distal end 1012, the proximal end of distal end 1012 prevents distal movement of proximal flaps 92 and, accordingly, inner tube 90 of inner shaft 82 relative to distal end 1012 beyond proximal flaps 92. Accordingly, the engagement between distal flaps 96 and counterbore 1014 and the engagement between proximal flaps 92 and the proximal end of distal end 1012 together releasably secure the engagement portion of inner shaft 82 to distal end 1012.

To decouple the engagement portion of inner shaft 82 from distal end 1012, outer tube 84 may be retracted proximally relative to inner tube 90, such that contact between the inner wall of the lumen of outer tube 84 and distal flaps 96 causes distal flaps 96 to transition to their collapsed position within receptacles 98 and remain in the collapsed position due to the constraint by the inner wall of the lumen of outer tube 84. With distal flaps 96 constrained within the lumen of outer tube 84, outer and inner tubes 84, 90 may be moved proximally relative to distal end 1012 to thereby decouple inner shaft 82 from distal end 1012. As will be understood by a person having ordinary skill in the art, the implantable occluder devices described herein may include distal end 1012 (and, accordingly the delivery system may include inner shaft 82), and any of the (threaded or threadless) proximal ends described above (and, according the delivery system may include a corresponding outer shaft).

While various illustrative embodiments of the invention are described above, it will be apparent to one skilled in the art that various changes and modifications may be made therein without departing from the invention. The appended claims are intended to cover all such changes and modifications that fall within the true scope of the invention.

What is claimed:

1. An occluder device for occluding a passage in a body, the occluder device comprising:
a plurality of filaments coupled to and extending between proximal and distal ends of the occluder device to define a proximal portion, a distal portion, and a central portion disposed between the proximal and distal portions, the proximal and distal ends configured to move relative to each other to cause the plurality of filaments to transition between a collapsed delivery state and an expanded deployed state where the proximal and distal portions expand radially outward; and
a biocompatible fabric patch configured to encapsulate at least the outer edges of the proximal and distal portions, the biocompatible fabric patch comprising a first inner membrane disposed between the proximal portion and the central portion, a second inner membrane disposed between the distal portion and the central portion, and an outer membrane disposed at the proximal and distal portions, each of the first and second inner membranes comprising a plurality of openings sized and shaped to receive the plurality of filaments therethrough,
wherein the proximal and distal ends are configured to lock together in a locked state to maintain the occluder device in the expanded deployed state, and
wherein the proximal portion is configured to be disposed within a first atrium, the central portion is configured to be disposed within an opening of an atrial septum, and the distal portion is configured to be disposed within a second atrium such that, in the expanded deployed state, the proximal and distal portions sandwich the atrial septum.

2. The occluder device of claim 1, wherein the plurality of filaments are arranged in a helical pattern about a longitudinal axis of the occluder device between the proximal and distal ends.

3. The occluder device of claim 1, wherein the plurality of filaments are bioresorbable.

4. The occluder device of claim 1, wherein at least one of the outer membrane, the first inner membrane, or the second inner membrane are formed of a bioresorbable material.

5. The occluder device of claim 1, further comprising a plurality of radiopaque markers disposed adjacent to the plurality of openings, the plurality of radiopaque markers coupled to at least one of the plurality of filaments, the first inner membrane, or the second inner membrane.

6. The occluder device of claim 5, wherein the plurality of radiopaque markers are disposed on both sides of the plurality of openings of at least one of the first or second inner membranes in an alternating manner.

7. The occluder device of claim 1, wherein the proximal end is configured to be removably coupled to an engagement portion at a distal region of an outer shaft of a delivery system, wherein the distal end is configured to be removably coupled to an engagement portion at a distal region of an inner shaft slidably disposed within the outer shaft, such that relative movement between the distal regions of the inner and outer shafts causes the occluder device to transition between the collapsed delivery state and the expanded deployed state.

8. The occluder device of claim 7, wherein the proximal end and the distal end are configured to be reattachably coupled to the distal regions of the outer shaft and the inner shaft, respectively, in situ.

9. The occluder device of claim 7, wherein a proximal region of the proximal end comprises:
a stopper extending radially outwardly and circumferentially about the outer surface of the proximal end; and
a plurality of radially outwardly extending protrusions disposed circumferentially along the outer surface of the proximal end distal to the stopper, the plurality of radially outwardly extending protrusions separated by a plurality of openings extending between the outer surface of the proximal end and a lumen of the proximal end, the plurality of openings sized and shaped to releasably receive the engagement portion of the outer shaft when the engagement portion of the outer shaft is inserted through the lumen of the proximal end and aligned with the plurality of openings to thereby removably couple the proximal end to the outer shaft,
wherein the stopper prevents proximal movement of the outer shaft relative to the proximal end and the plurality of openings prevent distal movement of the outer shaft relative to the proximal end.

10. The occluder device of claim 9, wherein the engagement portion of the outer shaft comprises a plurality of longitudinally extending arms configured to bend radially inward from a non-bended state for insertion through the lumen of the proximal end, each of the longitudinally extending arms comprising a distal protrusion configured to be releasably received through the plurality of openings of the proximal end in the non-bended state to thereby removably couple the proximal end to the outer shaft, and
wherein a lumen of the outer shaft is sized and shaped to slidably receive a core therethrough, the core configured to prevent the plurality of longitudinally extending arms from bending radially inward from the non-bended state.

11. The occluder device of claim 10, wherein the core comprises the inner shaft.

12. The occluder device of claim 7, wherein a lumen of the distal end comprises a counterbore extending circumferentially along an inner surface of the lumen, the counterbore sized and shaped to releasably receive the engagement portion of the inner shaft when the engagement portion of the inner shaft is inserted through the lumen of the distal end and aligned with the counterbore to thereby removably couple the distal end to the inner shaft, and
wherein a proximal side of the counterbore comprises a tapered profile configured to facilitate decoupling of the engagement portion of the inner shaft from the distal end.

13. The occluder device of claim 12, wherein the engagement portion of the inner shaft comprises a plurality of longitudinally extending arms configured to bend radially inward from a non-bended state for insertion through the lumen of the distal end, each of the longitudinally extending arms comprising a distal protrusion configured to be releasably received within the counterbore of the distal end in the non-bended state to thereby removably couple the distal end to the inner shaft,
wherein a lumen of the inner shaft is sized and shaped to slidably receive a core therethrough, the core configured to prevent the plurality of longitudinally extending arms from bending radially inward from the non-bended state, and
wherein the core comprises a guidewire tube or a guidewire.

14. The occluder device of claim 13, wherein, when the core is disposed within the lumen of the inner shaft, a proximal side of the counterbore prevents proximal movement of the inner shaft relative to the distal end and a distal side of the counterbore prevents distal movement of the inner shaft relative to the distal end.

15. The occluder device of claim 13, wherein the engagement portion of the inner shaft further comprises a plurality of flaps disposed circumferentially along an outer surface of the engagement portion proximal to the plurality of longitudinally extending arms, the plurality of flaps configured to transition between a collapsed position where the plurality of flaps extend parallel to a longitudinal axis of the inner shaft, and an expanded position where free distal ends of the plurality of flaps extend radially outward in the distal direction, the plurality of flaps biased towards the expanded position, and
wherein, when the core is disposed within the lumen of the inner shaft, a proximal side of the counterbore prevents proximal movement of the inner shaft relative to the distal end, and engagement between the plurality of flaps in the expanded position and a proximal side of the distal end prevents distal movement of the inner shaft relative to the distal end.

16. The occluder device of claim 12, wherein the engagement portion of the inner shaft comprises an outer tube comprising a plurality of longitudinally extending slits, and an inner tube slidably disposed within a lumen of the outer tube, the inner tube comprising:
a plurality of distal flaps disposed circumferentially along an outer surface of the inner tube, the plurality of distal flaps configured to transition between a collapsed position where the plurality of distal flaps extend parallel to a longitudinal axis of the inner shaft, and an expanded position where free proximal ends of the plurality of distal flaps extend radially outward through the plurality of slits in the proximal direction, the plurality of distal flaps biased towards the expanded position; and
a plurality of proximal flaps disposed circumferentially along an outer surface of the inner tube proximal to the plurality of distal flaps, the plurality of proximal flaps configured to transition between a collapsed position where the plurality of proximal flaps extend parallel to a longitudinal axis of the inner shaft, and an expanded position where free distal ends of the plurality of proximal flaps extend radially outward through the plurality of slits in the distal direction, the plurality of proximal flaps biased towards the expanded position,
wherein movement of the outer tube proximally relative to the inner tube causes the plurality of distal flaps to transition towards the collapsed position within the outer tube, and movement of the outer tube distally relative to the inner tube causes the plurality of proximal flaps to transition towards the collapsed position within the outer tube,
wherein, when the plurality of distal flaps are disposed within the counterbore of the distal end in the expanded position, a proximal side of the counterbore prevents proximal movement of the inner tube relative to the distal end, and engagement between the plurality of proximal flaps in the expanded position and a proximal side of the distal end prevents distal movement of the inner tube relative to the distal end,
wherein, when the core is disposed within the lumen of the inner shaft, a proximal side of the counterbore prevents proximal movement of the inner shaft relative to the distal end and a distal side of the counterbore prevents distal movement of the inner shaft relative to the distal end.

17. The occluder device of claim 7, wherein a proximal region of the proximal end comprises:
a proximal stopper extending radially outwardly and circumferentially about the outer surface of the proximal end;
a distal stopper extending radially outwardly and circumferentially about the outer surface of the proximal end; and
a gap disposed between the distal stopper and the proximal stopper, the gap configured to releasably receive the engagement portion of the outer shaft to thereby removably couple the proximal end to the outer shaft,
wherein the engagement portion of the outer shaft comprises a plurality of longitudinally extending arms configured to bend radially outward from a non-bended state for advancement over the proximal stopper of the proximal end, each of the longitudinally extending arms comprising a distal hook portion comprising a tooth configured to be received within the gap and a receptacle configured to receive at least a portion of the proximal stopper therein to thereby removably couple the proximal end to the outer shaft,
wherein the plurality of longitudinally extending arms are configured to be constrained in the non-bended state via an external sleeve slidably disposed over the engagement portion of the outer shaft, and
wherein, when the teeth of the engagement portion of the outer shaft is received within the gap, the proximal stopper prevents proximal movement of the outer shaft relative to the proximal end and the distal stopper prevents distal movement of the outer shaft relative to the proximal end.

18. The occluder device of claim 17, wherein the proximal end further comprises a plurality of planar surfaces disposed circumferentially about the outer surface of the proximal end within the gap, the plurality of planar surfaces configured to facilitate transmission of torque from the engagement portion of the outer shaft to the proximal end when the teeth of the engagement portion of the outer shaft is received within the gap.

19. The occluder device of claim 1, wherein the outer membrane comprises a continuous outer cover configured to be disposed over the proximal portion, the central portion, and the distal portion.

20. The occluder device of claim 19, wherein the continuous outer cover is only fixed to the occluder device via the proximal and distal ends of the occluder device.

21. The occluder device of claim 19, wherein the outer cover is formed by a thin weaved tubing, a knitted fabric, or an electro-spun polymer, and
wherein the electro-spun polymer comprises polytetrafluoroethylene (PTFE), polyethylene terephthalate (PET), or polyurethane (PU).

22. The occluder device of claim 19, wherein proximal and distal ends of the outer cover are configured to be coupled to the occluder device via at least one of mechanical crimping, chemical bonding, thermal bonding, or suturing.

23. The occluder device of claim 19, wherein at least one of the first or second inner membranes comprise a central aperture and a plurality of hollowed openings disposed circumferentially about the central aperture, the plurality of hollowed openings separated by a plurality of radially extending spokes and configured to improve membrane crossability.

24. The occluder device of claim 23, further comprising a plurality of radiopaque markers disposed adjacent to the plurality of openings, the plurality of radiopaque markers arranged to facilitate visualization of at least one recross zone sized and shaped to improve membrane crossability, the at least one recross zone aligned with at least one hollowed opening of the plurality of hollowed openings.

25. The occluder device of claim 19, wherein at least one of the first or second inner membranes comprise a full disk shape having a central aperture.

26. The occluder device of claim 1, wherein the outer membrane comprises:
a first outer membrane disposed on the proximal facing side of the proximal portion, an outer edge portion of the first outer membrane configured to be coupled to the outer perimeter of the first inner membrane; and
a second outer membrane disposed on the distal facing side of the distal portion, an outer edge portion of the second outer membrane configured to be coupled to the outer perimeter of the second inner membrane,
wherein the first and second outer membranes are coupled to the first and second inner membranes at a location radially inward from the outer edges of the proximal and distal portions, respectively, to reduce trauma to surrounding anatomy.

27. The occluder device of claim 26, wherein only a first subset of the plurality of filaments are coupled to the first outer membrane at the proximal portion, and wherein only a second subset of the plurality of filaments are coupled to the second outer membrane at the distal portion to thereby reduce global stiffness of the occluder device.

28. The occluder device of claim 26, wherein the first and second inner membranes are sized and shaped to fold over the outer edges of the proximal and distal portions, respectively, such that an outer perimeter of the first inner membrane is disposed on a proximal facing side of the proximal portion and an outer perimeter of the second inner membrane is disposed on a distal facing side of the distal portion.

29. The occluder device of claim 26, wherein each of the first and second inner membranes comprises a plurality of tabs disposed circumferentially along its outer perimeter, the plurality of tabs configured to extend radially inward, and
wherein each of the plurality of tabs comprises a plurality of guiding holes sized and shaped to facilitate suturing of the first and second inner membranes to the first and second outer membranes, respectively.

30. The occluder device of claim 26, wherein the first and second outer membranes are sized and shaped to fold over the outer edges of the proximal and distal portions, respectively, such that an outer perimeter of the first outer membrane is disposed on a distal facing side of the proximal portion and an outer perimeter of the second outer membrane is disposed on a proximal facing side of the distal portion.
